(19) United States
(12) Patent Application Publication
(10) Pub. No.: US 2026/0210966 A1
(43) Pub. Date: Jul. 23, 2026

(54) METHOD FOR PREDICTING FORMULATION EXHIBITING TARGET SENSORY QUALITY, AND METHOD FOR RECONFIGURING TARGET SENSORY QUALITY

(71) Applicant: Ajinomoto Co., Inc., Tokyo (JP)

(72) Inventors: Yusuke IHARA, Kanagawa (JP); Chiori IJICHI, Kanagawa (JP); Tetsuya KOBAYASHI, Tokyo (JP)

(73) Assignee: Ajinomoto Co., Inc., Tokyo (JP)

(21) Appl. No.: 19/570,289

(22) Filed: Mar. 18, 2026

Related U.S. Application Data
(63) Continuation of application No. PCT/JP2024/033769, filed on Sep. 20, 2024.

(30) Foreign Application Priority Data
Sep. 22, 2023 (JP) 2023-156650

(51) Int. Cl. G01N 33/68

(52) U.S. Cl. CPC G01N 33/68; G01N 2333/705

(57) ABSTRACT

A method of predicting a formulation that exhibits a target sensory quality, and a method of reconstructing a target sensory quality by using the method are provided. By identifying two or more substances and a combination ratio of receptor activity profiles of the substances such that the substances and the combination ratio provide a composite profile that approximates a receptor activity profile corresponding to a target sensory quality, a formulation that exhibits the target sensory quality is predicted, and further, the target sensory quality is reconstructed based on the predicted formulation.

This application is a Continuation of, and claims priority under 35 U.S.C. § 120 to, International Application No. PCT/JP2024/033769, filed Sep. 20, 2024, and claims priority therethrough under 35 U.S.C. § 119 to Japanese Patent Application No. 2023-156650, filed Sep. 22, 2023, the entireties of which, as well as all citations cited herein, are incorporated by reference herein.

### TECHNICAL FIELD

The present invention relates to a method of predicting a formulation that exhibits a target sensory quality, and a method of reconstructing the target sensory quality by using the method.

### BACKGROUND ART

Sensory qualities such as aroma and taste are important factors that influence the palatability of foods, fragrance/cosmetic products, and the like. Therefore, techniques for screening components required to reproduce a specific sensory quality, and techniques for reproducing a specific sensory quality by combining a plurality of components, are industrially important techniques for developing foods, fragrance/cosmetic products, and the like.

Conventionally, screening of components that affect a specific sensory quality has been carried out by having a human evaluate the sensory quality of a test substance through a sensory test. However, sensory tests have problems in, for example, that training of experts capable of evaluating sensory quality is required and that the tests have low throughput.

In methods that have been recently reported (for example, Patent Documents 1 and 2), responses of receptors such as olfactory receptors and taste receptors are used as indices to screen for substances that exhibit sensory qualities such as a specific aroma or taste.

Further, a method has been reported in which a formulation is optimized such that a synthesized spectrum obtained by combining MS spectra of a plurality of components approximates an MS spectrum of a target article that exhibits a target aroma, to reproduce the target aroma (for example, Non-Patent Document 1). However, in this method, only chemical structures contained in the target article that exhibit the target aroma can be imitated, and the target aroma cannot be reproduced by blending components having different chemical structures.

Further, a method has been reported (Non-Patent Document 2) in which a formulation is optimized such that a synthesized vector obtained, after correction with odor intensity, by combining 21 types of physicochemical feature vectors of a plurality of components approximates 21 types of physicochemical feature vectors of a target article that exhibits the target aroma, to reproduce the target aroma. However, in this method, it remains unclear whether the 21 types of physicochemical feature vectors sufficiently reflect the aroma. Further, in this method, information on odor intensity is required, so that a sensory test is necessary, and there is room for improvement in throughput and accuracy.

### PRIOR ART DOCUMENTS

### Patent Documents

[Patent Document 1] JP 2019-037197 A
[Patent Document 2] JP 2018-014999 A

### Non-Patent Documents

[Non-Patent Document 1] Dani Prasetyawan and Nakamoto Takamichi, Sensory Evaluation of Odor Approximation Using NMF with Kullback-Leibler Divergence and Itakura-Saito Divergence in Mass Spectrum Space, Journal of The Electrochemical Society, 2020 J. Electrochem. Soc. 167 167520.
[Non-Patent Document 2] Aharon Ravia et al., A measure of smell enables the creation of olfactory metamers, Nature volume 588, pages 118-123 (2020)

### SUMMARY OF THE INVENTION

An object of the present invention is to provide a method of predicting a formulation that exhibits a target sensory quality, and a method of reconstructing a target sensory quality by using the method.

As a result of intensive study to solve the above problem, the present inventors discovered that, by identifying two or more substances and a combination ratio of receptor activity profiles of the substances such that the substances and the combination ratio provide a composite profile that approximates a receptor activity profile corresponding to a target sensory quality, a formulation that exhibits the target sensory quality can be predicted, and also discovered that the target sensory quality can be reconstructed based on the predicted formulation, thereby completing the present invention.

Specifically, the present invention can be exemplified as follows.

[1]

A method of predicting a formulation that exhibits a target sensory quality, the method comprising the step of:
identifying two or more target substances and a combination ratio of receptor activity profiles of the target substances such that the target substances and the combination ratio provide a composite profile that approximates a target profile,
wherein each of the receptor activity profiles is a receptor activity profile for two or more receptors,
wherein the target profile is a receptor activity profile corresponding to the target sensory quality for the receptors, and
wherein a formulation of the target substances with the combination ratio is regarded as the formulation that exhibits the target sensory quality.
   [2]

The method (specifically, the method according to [1]), wherein the target substances comprise 2 to 8 types of substances.

[3]

The method (specifically, the method according to [1] or [2]), wherein, in the step, the target substances are selected from a test substance group.

[4]

The method (specifically, the method according to [3]), wherein the test substance group comprises more types of substances than the target substances.

[5]

The method (specifically, the method according to [3] or [4]), wherein the test substance group comprises not less than 100 types of substances.

[6]

The method (specifically, the method according to any one of [1] to [5]), wherein, in the step, the target profile is used as an objective variable, and the receptor activity profile, for the receptors, of each substance constituting the test substance group is used as an explanatory variable, and wherein the step is carried out by nonnegative constraint sparse modeling.

[7]

The method (specifically, the method according to [6]), wherein the sparse modeling is carried out using a LASSO regression model.

[8]

The method (specifically, the method according to any one of [1] to [7]), wherein the step is carried out such that an error between the target profile and the composite profile is not more than 0.7 in terms of a weighted RMSE.

[9]

The method (specifically, the method according to any one of [1] to [8]), wherein the receptors comprise not less than 300 types of receptors.

[10]

The method (specifically, the method according to any one of [1] to [9]), wherein the receptors are selected from the group consisting of G protein-coupled receptors and ion channel-type receptors.

[11]

The method (specifically, the method according to any one of [1] to [10]), wherein the receptors are selected from the group consisting of olfactory receptors and taste receptors.

[12]

The method (specifically, the method according to any one of [1] to [11]), wherein not less than 50% of the total number of receptors are selected from the group consisting of OR1A1, OR1A2, OR1B1, OR1C1, OR1D2, OR1D5, OR1E1, OR1F1, OR1F12, OR1G1, OR1I1, OR1J1, OR1J2, OR1J4, OR1K1, OR1L1, OR1L3, OR1L4, OR1L8, OR1M1, OR1N1, OR1N2, OR1Q1, OR1R1P, OR1S1, OR2A1, OR2A2, OR2A4, OR2A5, OR2A12, OR2A14, OR2A25, OR2AE1, OR2AG1, OR2AG2, OR2AJ1P, OR2AK2, OR2AP1, OR2AT4, OR2B2, OR2B3, OR2B6, OR2B11, OR2C1, OR2C3, OR2D2, OR2D3, OR2F1, OR2G2, OR2G3, OR2G6, OR2H1, OR2H2, OR2J2, OR2J3, OR2K2, OR2L2, OR2L8, OR2L13, OR2M2, OR2M4, OR2M7, OR2S2, OR2T1, OR2T2, OR2T5, OR2T6, OR2T8, OR2T10, OR2T11, OR2T27, OR2T34, OR2V2, OR2W1, OR2W3, OR2Y1, OR2Z1, OR3A1, OR3A2, OR3A3, OR3A4, OR4A5, OR4A15, OR4A16, OR4A47, OR4B1, OR4C3, OR4C5, OR4C6, OR4C11, OR4C12, OR4C13, OR4C15, OR4C16, OR4C46, OR4D1, OR4D2, OR4D5, OR4D6, OR4D9, OR4D10, OR4D11, OR4E2, OR4F3, OR4F5, OR4F6, OR4F14P, OR4F15, OR4G11P, OR4H12P, OR4K1, OR4K2, OR4K5, OR4K13, OR4K14, OR4K15, OR4K17, OR4L1, OR4M1, OR4N2, OR4N4, OR4N5, OR4P4, OR4Q3, OR4S1, OR4S2, OR4X1, OR4X2, OR5A1, OR5A2, OR5AC2, OR5AK2, OR5AK3P, OR5AN1, OR5AP2, OR5AR1, OR5AS1, OR5AU1, OR5B2, OR5B3, OR5B12, OR5B17, OR5B21, OR5C1, OR5D13, OR5D14, OR5D16, OR5D18, OR5F1, OR5H1, OR5H2, OR5H6, OR5H14, OR511, OR5J2, OR5K1, OR5K3, OR5K4, OR5L2, OR5M3, OR5M8, OR5M9, OR5M10, OR5M11, OR5P3, OR5R1, OR5T1, OR5T2, OR5T3, OR5V1, OR5W2, OR6A2, OR6B1, OR6B2, OR6C1, OR6C2, OR6C3, OR6C4, OR6C6, OR6C65, OR6C66P, OR6C68, OR6C70, OR6C74, OR6C75, OR6C76, OR6F1, OR6J1, OR6K2, OR6K3, OR6K6, OR6M1, OR6N1, OR6N2, OR6P1, OR6Q1, OR6S1, OR6T1, OR6V1, OR6X1, OR6Y1, OR7A3P, OR7A5, OR7A10, OR7A17, OR7C1, OR7C2, OR7D2, OR7D4, OR7E24, OR7G1, OR7G2, OR7G3, OR8A1, OR8B3, OR8B4, OR8B8, OR8B12, OR8D1, OR8D2, OR8D4, OR8G2, OR8G5, OR8H3, OR8I2, OR8J1, OR8J3, OR8K1, OR8K3, OR8K5, OR8S1, OR8U1, OR9A4, OR9G1, OR9G4, OR9I1, OR9K2, OR9Q1, OR9Q2, OR10A3, OR10A4, OR10A5, OR10A6, OR10A7, OR10AD1, OR10AG1, OR10C1, OR10D3, OR10D4P, OR10G2, OR10G3, OR10G4, OR10G6, OR10G7, OR10G9, OR10H2, OR10H4, OR10J1, OR10J3, OR10J5, OR10K1, OR10K2, OR10P1, OR10Q1, OR10R2, OR10S1, OR10T2, OR10V1, OR10W1, OR10X1, OR10Z1, OR11A1, OR11G2, OR11H4, OR11H6, OR11H12, OR11L1, OR12D2, OR12D3, OR13A1, OR13C2, OR13C3, OR13C4, OR13C8, OR13D1, OR13F1, OR13G1, OR13H1, OR13J1, OR14A2, OR14A16, OR14C36, OR1411, OR14J1, OR14K1, OR14L1P, OR51A1P, OR51A4, OR51A7, OR51B2, OR51B4, OR51B5, OR51B6, OR51D1, OR51E1, OR51E2, OR51F1, OR51F2, OR51F5P, OR51G1, OR51G2, OR51H1, OR51I1, OR51I2, OR51L1, OR51M1, OR51Q1, OR51S1, OR51T1, OR51V1, OR52A1, OR52A4, OR52A5, OR52B2, OR52B4, OR52B6, OR52D1, OR52E2, OR52E4, OR52E5, OR52E8, OR52H1, OR5212, OR52J3, OR52K2, OR52L2P, OR52M1, OR52N1, OR52N2, OR52N4, OR52N5, OR52P2P, OR52R1, OR52W1, OR52Z1P, OR56A1, OR56A3, OR56A4, OR56A5, OR56B1, OR56B2P, and OR56B4.

[13]

The method (specifically, the method according to any one of [1] to [12]), wherein the receptors comprise not less than 50% of OR1A1, OR1A2, OR1B1, OR1C1, OR1D2, OR1D5, OR1E1, OR1F1, OR1F12, OR1G1, OR1I1, OR1J1, OR1J2, OR1J4, OR1K1, OR1L1, OR1L3, OR1L4, OR1L8, OR1M1, OR1N1, OR1N2, OR1Q1, OR1R1P, OR1S1, OR2A1, OR2A2, OR2A4, OR2A5, OR2A12, OR2A14, OR2A25, OR2AE1, OR2AG1, OR2AG2, OR2AJ1P, OR2AK2, OR2AP1, OR2AT4, OR2B2, OR2B3, OR2B6, OR2B11, OR2C1, OR2C3, OR2D2, OR2D3, OR2F1, OR2G2, OR2G3, OR2G6, OR2H1, OR2H2, OR2J2, OR2J3, OR2K2, OR2L2, OR2L8, OR2L13, OR2M2, OR2M4, OR2M7, OR2S2, OR2T1, OR2T2, OR2T5, OR2T6, OR2T8, OR2T10, OR2T11, OR2T27, OR2T34, OR2V2, OR2W1, OR2W3, OR2Y1, OR2Z1, OR3A1, OR3A2, OR3A3, OR3A4, OR4A5, OR4A15, OR4A16, OR4A47, OR4B1, OR4C3, OR4C5, OR4C6, OR4C11, OR4C12, OR4C13, OR4C15, OR4C16, OR4C46, OR4D1, OR4D2, OR4D5, OR4D6, OR4D9, OR4D10, OR4D11, OR4E2, OR4F3, OR4F5, OR4F6, OR4F14P, OR4F15, OR4G11P, OR4H12P, OR4K1, OR4K2, OR4K5, OR4K13, OR4K14, OR4K15, OR4K17, OR4L1, OR4M1, OR4N2, OR4N4, OR4N5, OR4P4, OR4Q3, OR4S1, OR4S2, OR4X1, OR4X2, OR5A1, OR5A2, OR5AC2, OR5AK2, OR5AK3P, OR5AN1, OR5AP2, OR5AR1, OR5AS1, OR5AU1, OR5B2, OR5B3, OR5B12, OR5B17, OR5B21, OR5C1, OR5D13, OR5D14, OR5D16, OR5D18, OR5F1, OR5H1, OR5H2, OR5H6, OR5H14, OR511, OR5J2, OR5K1, OR5K3, OR5K4, OR5L2, OR5M3, OR5M8, OR5M9, OR5M10, OR5M11, OR5P3, OR5R1, OR5T1, OR5T2, OR5T3, OR5V1, OR5W2, OR6A2, OR6B1, OR6B2, OR6C1, OR6C2, OR6C3, OR6C4, OR6C6, OR6C65, OR6C66P, OR6C68, OR6C70, OR6C74, OR6C75, OR6C76, OR6F1, OR6J1, OR6K2, OR6K3, OR6K6, OR6M1, OR6N1, OR6N2, OR6P1, OR6Q1, OR6S1, OR6T1, OR6V1, OR6X1, OR6Y1, OR7A3P, OR7A5, OR7A10, OR7A17, OR7C1, OR7C2, OR7D2, OR7D4, OR7E24, OR7G1, OR7G2, OR7G3, OR8A1, OR8B3, OR8B4, OR8B8, OR8B12, OR8D1, OR8D2, OR8D4, OR8G2, OR8G5, OR8H3, OR8I2, OR8J1, OR8J3, OR8K1, OR8K3, OR8K5, OR8S1, OR8U1, OR9A4, OR9G1, OR9G4, OR9I1, OR9K2, OR9Q1, OR9Q2, OR10A3, OR10A4, OR10A5, OR10A6, OR10A7, OR10AD1, OR10AG1, OR10C1, OR10D3, OR10D4P, OR10G2, OR10G3, OR10G4, OR10G6, OR10G7, OR10G9, OR10H2, OR10H4, OR10J1, OR10J3, OR10J5, OR10K1, OR10K2, OR10P1, OR10Q1, OR10R2, OR10S1, OR10T2, OR10V1, OR10W1, OR10X1, OR10Z1, OR11A1, OR11G2, OR11H4, OR11H6, OR11H12, OR11L1, OR12D2, OR12D3, OR13A1, OR13C2, OR13C3, OR13C4, OR13C8, OR13D1, OR13F1, OR13G1, OR13H1, OR13J1, OR14A2, OR14A16, OR14C36, OR1411, OR14J1, OR14K1, OR14L1P, OR51A1P, OR51A4, OR51A7, OR51B2, OR51B4, OR51B5, OR51B6, OR51D1, OR51E1, OR51E2, OR51F1, OR51F2, OR51F5P, OR51G1, OR51G2, OR51H1, OR51I1, OR51I2, OR51L1, OR51M1, OR51Q1, OR51S1, OR51T1, OR51V1, OR52A1, OR52A4, OR52A5, OR52B2, OR52B4, OR52B6, OR52D1, OR52E2, OR52E4, OR52E5, OR52E8, OR52H1, OR5212, OR52J3, OR52K2, OR52L2P, OR52M1, OR52N1, OR52N2, OR52N4, OR52N5, OR52P2P, OR52R1, OR52W1, OR52Z1P, OR56A1, OR56A3, OR56A4, OR56A5, OR56B1, OR56B2P, and OR56B4.

[14]

The method (specifically, the method according to any one of [1] to [13]), wherein the receptors are human receptors.

[15]

The method (specifically, the method according to any one of [1] to [14]), further comprising the step of evaluating a sensory quality of the predicted formulation.

[16]

The method (specifically, the method according to [15]), wherein the evaluation is carried out by sensory evaluation.

[17]

The method (specifically, the method according to any one of [1] to [16]), comprising:
a step of identifying, for each of a plurality of targets, the two or more target substances and the combination ratio of receptor activity profiles of the target substances such that the target substances and the combination ratio provide the composite profile that approximates the target profile; and
a minimum set determination step of determining a predetermined number of synthetic elements and combination ratios of the synthetic elements such that the synthetic elements and the combination ratios approximate the identified composite profiles of the plurality of targets, and determining one or more target substances that constitute each of the synthetic elements.
   [18]

The method (specifically, the method according to [17]), wherein, in the minimum set determination step, the composite profiles of the plurality of targets are decomposed, by nonnegative matrix factorization (NMF), into the predetermined number of synthetic elements and the combination ratios of the synthetic elements.

[19]

A method of reconstructing a target sensory quality, the method comprising the steps of:
predicting a formulation that exhibits a target sensory quality by the method (specifically, the method according to any one of [1] to [16]); and
blending the target substances based on the formulation.
   [20]

The method (specifically, the method according to [19]), wherein the target substances are blended at a blending ratio that is substantially identical to the combination ratio.

[21]

The method (specifically, the method according to [20]), wherein, for each target substance, the blending ratio is 0.9 to 1.1 times the combination ratio.

[22]

A method of reconstructing a target sensory quality, the method comprising the steps of:
determining a predetermined number of synthetic elements and combination ratios of the synthetic elements such that the synthetic elements and the combination ratios approximate the composite profiles of the plurality of targets, and determining one or more target substances that constitute each of the synthetic elements, by the method (specifically, the method according to [17] or [18]); and
blending the synthetic elements based on the combination ratios of the synthetic elements.

### Effect of the Invention

According to the present invention, a formulation that exhibits a target sensory quality can be predicted. Further, according to the present invention, the target sensory quality can be reconstructed based on the predicted formulation.

### BRIEF DESCRIPTION OF THE DRAWINGS

FIG. 1 is a diagram for illustrating reconstruction of a target set of interest.

FIG. 2 is a diagram illustrating a basis matrix A.

FIG. 3 is a diagram illustrating a coefficient matrix H.

FIG. 4 is a diagram illustrating a reconstruction recipe matrix W.

FIG. 5 is a diagram illustrating an approximation matrix AH.

FIG. 6 is a diagram illustrating a relationship between the number of fragrance cartridges in a minimum set and an approximation accuracy by the approximation matrix AH.

FIG. 7 is a diagram illustrating a basis matrix A.

FIG. 8 is a diagram illustrating a coefficient matrix H.

FIG. 9 is a diagram illustrating a target profile matrix W.

FIG. 10 is a diagram illustrating an approximation matrix AH.

### MODE FOR CARRYING OUT THE INVENTION

The present invention is described below in detail.

### <1> Method of the Present Invention

One aspect of the method of the present invention is a method of predicting a formulation that exhibits a target sensory quality. This method is also referred to as a “prediction method of the present invention.” A formulation that exhibits a target sensory quality is also referred to as a “target formulation.”

One aspect of the method of the present invention is a method of reconstructing a target sensory quality. This method is also referred to as a “reconstruction method of the present invention.”

### <1-1> Sensory Quality

The term “target sensory quality” means a sensory quality that is to be targeted in the method of the present invention. Specifically, the term “target sensory quality” means a sensory quality exhibited by a formulation that is to be predicted in the prediction method of the present invention. The term “target sensory quality” also specifically means a sensory quality that is to be reconstructed in the reconstruction method of the present invention. The type of target sensory quality is not particularly limited. Examples of the sensory quality include sensations perceived in the oral cavity and/or nasal cavity when a target article is used (for example, when the target article is eaten). The target article may be a target article composed of a single component (that is, a pure substance), or may be a target article composed of a combination of two or more components (that is, a mixture). Examples of the target article include aroma components and taste components, as well as foods and fragrance/cosmetic products containing these. Specific examples of the sensory quality include aroma, taste, and flavor. The target sensory quality may be a single type of sensory quality, or may be a combination of two or more types of sensory qualities.

Examples of the aroma include absinthe, acacia, acai, acerola, acetic, acetone, acidic, acorn, acrylate, agarwood, alcoholic, aldehydic, alfalfa, algae, alliaceous, allspice, almond, almond bitter almond, almond roasted almond, almond toasted almond, amber, ambergris, ambrette, ammoniacal, angelica, animal, anise, anisic, apple, apple cooked apple, apple dried apple, apple green apple, apple red apple, apple skin, apricot, aromatic, arrack, artichoke, asafetida, asparagus, astringent, autumn, avocado, bacon, baked, balsamic, banana, banana peel, banana ripe banana, banana unripe banana, barley roasted barley, basil, bay, bean green bean, beany, beef juice, beefy, beefy roasted beefy, beer, beeswax, benzoin, bergamot, berry, berry ripe berry, bitter, blackberry, bloody, blueberry, bois de rose, boronia, bouillon, boysenberry, brandy, bread baked, bread crust, bread rye bread, bready, broccoli, brothy, brown, bubble gum, buchu, burnt, butter rancid, buttermilk, butterscotch, buttery, cabbage, calamus, camphoreous, cananga, candy, cantaloupe, capers, caramellic, caraway, cardamom, carnation, carrot, carrot seed, carvone, cascarilla, cashew, cassia, castoreum, catty, cauliflower, cedar, cedarwood, celery, cereal, chamomile, charred, cheesy, cheesy bleu cheese, cheesy cheddar cheese, cheesy feta cheese, cheesy gorgonzola cheese, cheesy gouda cheese, cheesy limburger cheese, cheesy parmesan cheese, cheesy roquefort cheese, chemical, cherry, cherry maraschino cherry, chervil, chestnut, chicken, chicken coup, chicken fat, chicken roasted chicken, chicory, chive, chocolate, chocolate dark chocolate, chocolate white chocolate, chrysanthemum, cider, cilantro, cilantro, cinnamon, cinnamyl, cistus, citronella, citrus, citrus peel, citrus rind, civet, clam, clean, cloth laundered cloth, clove, clover, cocoa, coconut, coffee, coffee roasted coffee, cognac, cologne, cooked, cookie, cooling, copaiba, coriander, corn, corn chip, cornmeal, cornmint, cortex, costus, cotton candy, coumarinic, cranberry, creamy, cubeb, cucumber, cucumber skin, cumin, currant black currant, currant bud black currant bud, currant red currant, curry, custard, cyclamen, cypress, dairy, date, davana, deertongue, dewy, dill, dirty, dragon fruit, dry, durian, dusty, earthy, egg nog, egg yolk, eggy, elderberry, elderflower, elemi, estery, ethereal, eucalyptus, fatty, fecal, fennel, fenugreek, fermented, fig, filbert, fir needle, fishy, fleshy, floral, foliage, forest, fougere, frankincense, freesia, fresh, fresh outdoors, fried, fruit dried fruit, fruit overripe fruit, fruit ripe fruit, fruit tropical fruit, fruity, fudge, fungal, fusel, galanga, galbanum, gardenia, garlic, gasoline, gassy, genet, geranium, ginger, ginseng, goaty, goji berry, gooseberry, gourmand, graham cracker, grain, grain toasted grain, grape, grape skin, grapefruit, grapefruit peel, grassy, gravy, greasy, green, grilled, guaiacol, guaiacwood, guava, hairy, ham, harsh, hawthorn, hay, hay new mown hay, hazelnut, hazelnut roasted hazelnut, heather, heliotrope, herbal, hibiscus, honey, honeydew, honeysuckle, hops, horehound, horseradish, huckleberry, humus, hyacinth, hyssop, immortelle, incense, jackfruit, jammy, jasmin, jonquil, juicy, juicy fruit, juniper, ketonic, kimchi, kiwi, kokumi, kumquat, labdanum, lachrymatory, lactonic, lamb, lard, lavandin, lavender, lavender spike lavender, leafy, leathery, leek, lemon, lemon peel, lemongrass, lettuce, licorice, licorice black licorice, lilac, lily, lily of the valley, lime, linden flower, lingonberry, liver, lobster, loganberry, lovage, lychee, macadamia, mace, magnolia, mahogany, malty, mandarin, mango, maple, marigold, marine, marjoram, marshmallow, marzipan, mastic, meaty, meaty roasted meaty, medicinal, melon, melon rind, melon unripe melon, mentholic, metallic, milky, mimosa, minty, molasses, moldy, mossy, muguet, mulberry, mushroom, musk, mustard, musty, mutton, myrrh, naphthyl, narcissus, nasturtium, natural, neroli, noni fruit, nut flesh, nut skin, nutmeg, nutty, oakmoss, oatmeal, oats, ocean, oily, onion, onion cooked onion, onion green onion, opoponax, orange, orange bitter orange, orange peel, orange rind, orangeflower, orchid, oriental, origanum, orris, osmanthus, oyster, ozone, painty, palmarosa, papaya, paper, parsley, passion fruit, patchouli, pea green pea, peach, peanut, peanut butter, peanut roasted peanut, pear, pear skin, pecan, peely, pennyroyal, peony, pepper bell pepper, pepper black pepper, peppermint, peppery, peru balsam, petal, petitgrain, petroleum, phenolic, pimenta, pine, pineapple, pistachio, plastic, plum, plum skin, pomegranate, popcorn, pork, potato, potato baked potato, potato chip, potato raw potato, powdery, praline, privet, privetblossom, prune, pulpy, pumpkin, pungent, quince, radish, rain, raisin, rancid, raspberry, raw, reseda, resinous, rhubarb, rindy, ripe, roasted, root beer, rooty, rose, rose dried rose, rose red rose, rose tea rose, rose white rose, rosemary, rubbery, rue, rummy, saffron, sage, sage clary sage, salmon, salty, sandalwood, sandy, sappy, sarsaparilla, sassafras, sauerkraut, sausage, sausage smoked sausage, savory, sawdust, scallion, seafood, seashore, seaweed, seedy, sesame, sharp, shellfish, shrimp, skunk, smoky, soapy, soft, solvent, soup, sour, spearmint, spicy, spinach, spruce, starchy, starfruit, storax, strawberry, stringent, styrene, sugar, sugar brown sugar, sugar burnt sugar, sulfurous, sweaty, sweet, sweet pea, taco, tagette, tallow, tamarind, tangerine, tansy, tarragon, tart, tea, tea black tea, tea green tea, tea rooibos tea, tea white tea, tequila, terpenic, thujonic, thyme, toasted, tobacco, toffee, tolu balsam, tomato, tomato leaf, tonka, tropical, truffle, tuberose, tuna, turkey, turmeric, turnip, tutti frutti, umami, urine, valerian root, vanilla, vegetable, verbena, vetiver, vinegar, violet, violet leaf, walnut, warm, wasabi, watercress, watermelon, watermelon rind, watery, waxy, weedy, wet, whiskey, winey, wintergreen, woody, woody burnt wood, woody oak wood, woody old wood, wormwood, yeasty, ylang, yogurt, yuzu, zedoary, zesty, bark, birch bark, blood, raw meat, burnt candle, burnt milk, burnt pepper, burnt rubber, cadaverous (dead animal), cardboard, cat urine, chalky, cleaning fluid, cooked vegetables, cork, creosote, crushed grass, crushed weeds, dirty linen, disinfectant, carbolic, fermented (rotten) fruit, fragrant, fresh green vegetable, fresh tobacco smoke, fried chicken, heavy, household gas, kerosene, kippery (smoked fish), laurel leaves, light, mothballs, mouse, nail polish remover, new rubber, peanut butter, perfumery, putrid, four, decayde, rope, seasoning (for meat), seminal, sperm-like, sewer, sickening, sooty, sour milk, stale, stale tobacco smoke, tab, tea leaves, turpentine (pine oil), varnish, wet paper, wet wool, and wet dog.

Examples of the taste include sweet taste, salty taste, sour taste, bitter taste, umami, kokumi, and fat taste.

Examples of the flavor include flavors corresponding to various foods or raw materials thereof.

### <1-2> Prediction Method of the Present Invention

A target formulation can be predicted by identifying two or more target substances and a combination ratio of receptor activity profiles of the target substances such that the target substances and the combination ratio provide a composite profile that approximates a target profile. Thus, the method of the present invention may be a method of predicting a formulation that exhibits a target sensory quality, the method comprising the step of identifying two or more target substances and a combination ratio of receptor activity profiles of the target substances such that the target substances and the combination ratio provide a composite profile that approximates a target profile. This step is also referred to as a “identification step”. The combination ratio is also referred to as “combination ratio of target substances” or simply as “combination ratio”. The composite profile may also be referred to as “composite profile of target substances” or simply as “composite profile”. The term “composite profile” may also be used interchangeably with the term “reconstructed profile”. According to the prediction method of the present invention, in particular when compared to cases where a target formulation is predicted based on sensory evaluation, prediction of a target formulation is expected to be possible with high throughput and high accuracy.

The expression “two or more target substances and a combination ratio of receptor activity profiles of the target substances provide a composite profile that approximates a target profile” means that the composite profile obtained by combining the receptor activity profiles of the target substances at the combination ratio approximates the target profile.

The term “target profile” means a receptor activity profile corresponding to a target sensory quality. The term “receptor activity profile corresponding to a target sensory quality” may mean a receptor activity profile of a target article that exhibits a target sensory quality. The target article that exhibits the target sensory quality may be composed of a single component (that is, a pure substance), or may be composed of a combination of two or more components (that is, a mixture). In cases where the target article that exhibits the target sensory quality is a mixture, each individual component constituting the mixture may or may not exhibit the target sensory quality as long as the mixture exhibits the target sensory quality. Examples of the target article that exhibits the target sensory quality include aroma components, taste components, and foods or fragrance/cosmetic products containing these. The target article that exhibits the target sensory quality may be an actual target article, or may be a virtual target article. In other words, the target profile may be an actual receptor activity profile, or may be a virtual receptor activity profile. Thus, specifically, the target profile may be a receptor activity profile of an actual target article that exhibits the target sensory quality, or may be a virtual receptor activity profile that assumes the target sensory quality. The target profile may be known or unknown. In cases where the target profile is unknown, the target profile may be obtained, as appropriate, before the identification step is carried out. Thus, the method of the present invention may include a step of obtaining a target profile before the identification step is carried out. A target profile of an actual target article can be obtained, for example, by the later-described method of obtaining a receptor activity profile of a substance. Further, a virtual receptor activity profile can be constructed, for example, based on a correlation between a sensory quality and a receptor activity profile. Specifically, a virtual receptor activity profile can be constructed, for example, by: (1) constructing, for a plurality of target articles, a model that explains or predicts a target quality (for example, based on evaluation by a developer) or a preference score (for example, based on a consumer test) from receptor activity profiles; (2) using the model to perform inverse search for a receptor activity profile that provides a preferable target quality or preference score (for example, a receptor activity profile that maximizes the target quality or the preference score) by an analytical technique such as inverse problem analysis or simulation; and (3) setting the inversely searched receptor activity profile as the target profile.

The term “target substance” means each substance that is identified in the identification step. In the identification step, each target substance may be selected from a test substance group.

Each target substance may be composed of a single component (that is, a pure substance), or may be composed of a combination of two or more components (that is, a mixture).

The number of target substances is not less than 2. The number of target substances may be set, for example, to a number suitable for reconstructing the target sensory quality. For example, the number of target substances may be not less than 2, not less than 3, not less than 4, not less than 5, not less than 6, not less than 7, not less than 8, not less than 9, not less than 10, not less than 12, not less than 15, or not less than 20, and may be not more than 25, not more than 20, not more than 15, not more than 12, not more than 10, not more than 9, not more than 8, not more than 7, not more than 6, not more than 5, not more than 4, or not more than 3. The number of target substances may also be a consistent combination of these numbers. Specifically, for example, the number of target substances may be 2 to 3, 3 to 4, 4 to 5, 5 to 6, 6 to 7, 7 to 8, 8 to 9, 9 to 10, 10 to 12, 12 to 15, 15 to 20, or 20 to 25. Specifically, for example, the number of target substances may be 2 to 15, 2 to 10, 2 to 8, or 2 to 4.

The term “test substance group” means a group of substances used as candidates for the target substances. Each substance constituting the test substance group is also referred to as a “test substance.”

The test substances are not particularly limited as long as their receptor activity profiles can be used. Each test substance may be composed of a single component (that is, a pure substance), or may be composed of a combination of two or more components (that is, a mixture). In cases where the test substance is a mixture, the number of types of components constituting the mixture and the composition ratio thereof are not particularly limited. The test substances may be known substances, or may be novel substances. The test substances may be natural substances, or may be artificial substances. The test substances may be, for example, a compound library prepared by using a combinatorial chemistry technique. The test substances may be, for example, prototype products for reconstructing the target sensory quality. Examples of the test substances include aroma components, taste components, various other food components and fragrance/cosmetic product components, and foods and fragrance/cosmetic products containing these. Specific examples of the test substances include alcohols, ketones, aldehydes, ethers, esters, hydrocarbons, saccharides, organic acids, nucleic acids, amino acids, peptides, and various other organic or inorganic components. Examples of the test substances include, in particular, existing food additives. The term “existing food additives” means substances whose use as food additives has already been approved. The receptor activity profiles of the test substances may be known or unknown. In cases where the receptor activity profiles of the test substances are unknown, the receptor activity profiles of the test substances may be obtained as appropriate before the identification step is carried out. Thus, the method of the present invention may include a step of obtaining the receptor activity profiles of the test substances before the identification step is carried out. The receptor activity profiles of the test substances can be obtained, for example, by the later-described method of obtaining a receptor activity profile of a substance.

The number of test substances is 2 or more. The number of test substances is equal to or greater than the number of target substances. In particular, the number of test substances may be higher than the number of target substances. For example, the number of test substances may be not less than 2, not less than 5, not less than 10, not less than 20, not less than 50, not less than 100, not less than 200, not less than 500, not less than 1000, not less than 2000, not less than 5000, not less than 10,000, not less than 20,000, not less than 50,000, not less than 100,000, not less than 200,000, or not less than 500,000, and may be not more than 1,000,000, not more than 500,000, not more than 200,000, not more than 100,000, not more than 50,000, not more than 20,000, not more than 10,000, not more than 5000, not more than 2000, not more than 1000, not more than 500, not more than 200, not more than 100, not more than 50, not more than 20, not more than 10, or not more than 5. The number of test substances may also be a consistent combination of these numbers. In particular, the number of test substances may be not less than 50, not less than 100, not less than 200, not less than 500, not less than 1000, or not less than 2000. Specifically, for example, the number of test substances may be 2 to 5, 5 to 10, 10 to 20, 20 to 50, 50 to 100, 100 to 200, 200 to 500, 500 to 1000, 1000 to 2000, 2000 to 5000, 5000 to 10,000, 10,000 to 20,000, 20,000 to 50,000, 50,000 to 100,000, 100,000 to 200,000, 200,000 to 500,000, or 500,000 to 1,000,000. Specifically, for example, the number of test substances may be 2 to 1,000,000, 2 to 100,000, 2 to 10,000, 2 to 1000, 2 to 100, 100 to 1,000,000, 100 to 100,000, 100 to 10,000, 100 to 1000, 1000 to 1,000,000, 1000 to 100,000, or 1000 to 10,000.

The term “receptor activity profile” means data that indicate the presence or absence of, or the degree of, the property of activating or inactivating each receptor. Specifically, a “receptor activity profile” of a certain substance may mean data indicating whether or not the substance activates or inactivates a receptor, or indicating the degree to which the substance activates or inactivates a receptor. In addition, a “receptor activity profile” of a certain substance for two or more receptors may mean a pattern indicating, among the two or more receptors, which receptors the substance activates or inactivates and which receptors the substance does not activate or inactivate, or a pattern indicating the degree to which the substance activates or inactivates each receptor. A receptor activity profile for two or more receptors can be handled, for example, as an N-dimensional vector (wherein N is the number of receptors). The term “substance” in the description of a receptor activity profile may be read as “target article” or “component.” A receptor activity profile is also referred to as “receptor activation characteristics.”

As the receptor activity profile, a receptor activity profile for two or more receptors is used. The two or more receptors that are selected are common among the receptor activity profiles of the target substances, the receptor activity profiles of the test substances, and the target profile.

The receptors are not particularly limited. Examples of the receptors include G protein-coupled receptors and ion channel-type receptors. Examples of the receptors also include olfactory receptors and taste receptors. Each olfactory receptor can be an example of a G protein-coupled receptor. Each taste receptor can be an example of a G protein-coupled receptor or an ion channel-type receptor.

Examples of the olfactory receptors (which may be G protein-coupled receptors) include OR1A1, OR1A2, OR1B1, OR1C1, OR1D2, OR1D4, OR1D5, OR1E1, OR1E2, OR1F1, OR1F12, OR1G1, OR1I1, OR1J1, OR1J2, OR1J4, OR1K1, OR1L1, OR1L3, OR1L4, OR1L6, OR1L8, OR1M1, OR1N1, OR1N2, OR1Q1, OR1R1P, OR1S1, OR1S2, OR2A1, OR2A2, OR2A4, OR2A5, OR2A7, OR2A12, OR2A14, OR2A25, OR2AE1, OR2AG1, OR2AG2, OR2AJ1P, OR2AK2, OR2AP1, OR2AT4, OR2B2, OR2B3, OR2B6, OR2B11, OR2C1, OR2C3, OR2D2, OR2D3, OR2F1, OR2F2, OR2G2, OR2G3, OR2G6, OR2H1, OR2H2, OR2J1P, OR2J2, OR2J3, OR2K2, OR2L2, OR2L3, OR2L5, OR2L8, OR2L13, OR2M2, OR2M3, OR2M4, OR2M5, OR2M7, OR2S2, OR2T1, OR2T2, OR2T3, OR2T4, OR2T5, OR2T6, OR2T7, OR2T8, OR2T10, OR2T11, OR2T12, OR2T27, OR2T29, OR2T33, OR2T34, OR2T35, OR2V1, OR2V2, OR2W1, OR2W3, OR2Y1, OR2Z1, OR3A1, OR3A2, OR3A3, OR3A4, OR4A4P, OR4A5, OR4A15, OR4A16, OR4A47, OR4B1, OR4C3, OR4C5, OR4C6, OR4C11, OR4C12, OR4C13, OR4C15, OR4C16, OR4C45, OR4C46, OR4D1, OR4D2, OR4D5, OR4D6, OR4D9, OR4D10, OR4D11, OR4E2, OR4F3, OR4F4, OR4F5, OR4F6, OR4F14P, OR4F15, OR4F17, OR4F21, OR4G11P, OR4H12P, OR4K1, OR4K2, OR4K5, OR4K13, OR4K14, OR4K15, OR4K17, OR4L1, OR4M1, OR4M2, OR4N2, OR4N4, OR4N5, OR4P4, OR4Q3, OR4S1, OR4S2, OR4X1, OR4X2, OR5A1, OR5A2, OR5AC2, OR5AK2, OR5AK3P, OR5AN1, OR5AP2, OR5AR1, OR5AS1, OR5AU1, OR5B2, OR5B3, OR5B12, OR5B17, OR5B21, OR5C1, OR5D13, OR5D14, OR5D16, OR5D18, OR5F1, OR5H1, OR5H2, OR5H6, OR5H14, OR5H15, OR511, OR5J2, OR5K1, OR5K2, OR5K3, OR5K4, OR5L1, OR5L2, OR5M1, OR5M3, OR5M8, OR5M9, OR5M10, OR5M11, OR5P2, OR5P3, OR5R1, OR5T1, OR5T2, OR5T3, OR5V1, OR5W2, OR6A2, OR6B1, OR6B2, OR6B3, OR6C1, OR6C2, OR6C3, OR6C4, OR6C6, OR6C65, OR6C66P, OR6C68, OR6C70, OR6C74, OR6C75, OR6C76, OR6F1, OR6J1, OR6K2, OR6K3, OR6K6, OR6M1, OR6N1, OR6N2, OR6P1, OR6Q1, OR6S1, OR6T1, OR6V1, OR6X1, OR6Y1, OR7A3P, OR7A5, OR7A10, OR7A17, OR7C1, OR7C2, OR7D2, OR7D4, OR7E24, OR7G1, OR7G2, OR7G3, OR8A1, OR8B2, OR8B3, OR8B4, OR8B8, OR8B12, OR8D1, OR8D2, OR8D4, OR8G1, OR8G2, OR8G5, OR8H1, OR8H2, OR8H3, OR8I2, OR8J1, OR8J3, OR8K1, OR8K3, OR8K5, OR8S1, OR8U1, OR8U8, OR9A2, OR9A4, OR9G1, OR9G4, OR9I1, OR9K2, OR9Q1, OR9Q2, OR10A2, OR10A3, OR10A4, OR10A5, OR10A6, OR10A7, OR10AD1, OR10AG1, OR10C1, OR10D3, OR10D4P, OR10G2, OR10G3, OR10G4, OR10G6, OR10G7, OR10G8, OR10G9, OR10H1, OR10H2, OR10H3, OR10H4, OR10H5, OR10J1, OR10J3, OR10J5, OR10K1, OR10K2, OR10P1, OR10Q1, OR10R2, OR10S1, OR10T2, OR10V1, OR10W1, OR10X1, OR10Z1, OR11A1, OR11G2, OR11H1, OR11H2, OR11H4, OR11H6, OR11H12, OR11L1, OR12D2, OR12D3, OR13A1, OR13C2, OR13C3, OR13C4, OR13C5, OR13C8, OR13C9, OR13D1, OR13F1, OR13G1, OR13H1, OR13J1, OR14A2, OR14A16, OR14C36, OR1411, OR14J1, OR14K1, OR14L1P, OR51A1P, OR51A2, OR51A4, OR51A7, OR51B2, OR51B4, OR51B5, OR51B6, OR51D1, OR51E1, OR51E2, OR51F1, OR51F2, OR51F5P, OR51G1, OR51G2, OR51H1, OR51I1, OR51I2, OR51L1, OR51M1, OR51Q1, OR51S1, OR51T1, OR51V1, OR52A1, OR52A4, OR52A5, OR52B2, OR52B4, OR52B6, OR52D1, OR52E2, OR52E4, OR52E5, OR52E6, OR52E8, OR52H1, OR52I1, OR52I2, OR52J3, OR52K1, OR52K2, OR52L1, OR52L2P, OR52M1, OR52N1, OR52N2, OR52N4, OR52N5, OR52P2P, OR52R1, OR52W1, OR52Z1P, OR56A1, OR56A3, OR56A4, OR56A5, OR56B1, OR56B2P, and OR56B4 (these are also referred to as “Group A”).

Examples of the olfactory receptors (which may be G protein-coupled receptors) include, in particular, OR1A1, OR1A2, OR1B1, OR1C1, OR1D2, OR1D5, OR1E1, OR1F1, OR1F12, OR1G1, OR1I1, OR1J1, OR1J2, OR1J4, OR1K1, OR1L1, OR1L3, OR1L4, OR1L8, OR1M1, OR1N1, OR1N2, OR1Q1, OR1R1P, OR1S1, OR2A1, OR2A2, OR2A4, OR2A5, OR2A12, OR2A14, OR2A25, OR2AE1, OR2AG1, OR2AG2, OR2AJ1P, OR2AK2, OR2AP1, OR2AT4, OR2B2, OR2B3, OR2B6, OR2B11, OR2C1, OR2C3, OR2D2, OR2D3, OR2F1, OR2G2, OR2G3, OR2G6, OR2H1, OR2H2, OR2J2, OR2J3, OR2K2, OR2L2, OR2L8, OR2L13, OR2M2, OR2M4, OR2M7, OR2S2, OR2T1, OR2T2, OR2T5, OR2T6, OR2T8, OR2T10, OR2T11, OR2T27, OR2T34, OR2V2, OR2W1, OR2W3, OR2Y1, OR2Z1, OR3A1, OR3A2, OR3A3, OR3A4, OR4A5, OR4A15, OR4A16, OR4A47, OR4B1, OR4C3, OR4C5, OR4C6, OR4C11, OR4C12, OR4C13, OR4C15, OR4C16, OR4C46, OR4D1, OR4D2, OR4D5, OR4D6, OR4D9, OR4D10, OR4D11, OR4E2, OR4F3, OR4F5, OR4F6, OR4F14P, OR4F15, OR4G11P, OR4H12P, OR4K1, OR4K2, OR4K5, OR4K13, OR4K14, OR4K15, OR4K17, OR4L1, OR4M1, OR4N2, OR4N4, OR4N5, OR4P4, OR4Q3, OR4S1, OR4S2, OR4X1, OR4X2, OR5A1, OR5A2, OR5AC2, OR5AK2, OR5AK3P, OR5AN1, OR5AP2, OR5AR1, OR5AS1, OR5AU1, OR5B2, OR5B3, OR5B12, OR5B17, OR5B21, OR5C1, OR5D13, OR5D14, OR5D16, OR5D18, OR5F1, OR5H1, OR5H2, OR5H6, OR5H14, OR5I1, OR5J2, OR5K1, OR5K3, OR5K4, OR5L2, OR5M3, OR5M8, OR5M9, OR5M10, OR5M11, OR5P3, OR5R1, OR5T1, OR5T2, OR5T3, OR5V1, OR5W2, OR6A2, OR6B1, OR6B2, OR6C1, OR6C2, OR6C3, OR6C4, OR6C6, OR6C65, OR6C66P, OR6C68, OR6C70, OR6C74, OR6C75, OR6C76, OR6F1, OR6J1, OR6K2, OR6K3, OR6K6, OR6M1, OR6N1, OR6N2, OR6P1, OR6Q1, OR6S1, OR6T1, OR6V1, OR6X1, OR6Y1, OR7A3P, OR7A5, OR7A10, OR7A17, OR7C1, OR7C2, OR7D2, OR7D4, OR7E24, OR7G1, OR7G2, OR7G3, OR8A1, OR8B3, OR8B4, OR8B8, OR8B12, OR8D1, OR8D2, OR8D4, OR8G2, OR8G5, OR8H3, OR8I2, OR8J1, OR8J3, OR8K1, OR8K3, OR8K5, OR8S1, OR8U1, OR9A4, OR9G1, OR9G4, OR9I1, OR9K2, OR9Q1, OR9Q2, OR10A3, OR10A4, OR10A5, OR10A6, OR10A7, OR10AD1, OR10AG1, OR10C1, OR10D3, OR10D4P, OR10G2, OR10G3, OR10G4, OR10G6, OR10G7, OR10G9, OR10H2, OR10H4, OR10J1, OR10J3, OR10J5, OR10K1, OR10K2, OR10P1, OR10Q1, OR10R2, OR10S1, OR10T2, OR10V1, OR10W1, OR10X1, OR10Z1, OR11A1, OR11G2, OR11H4, OR11H6, OR11H12, OR11L1, OR12D2, OR12D3, OR13A1, OR13C2, OR13C3, OR13C4, OR13C8, OR13D1, OR13F1, OR13G1, OR13H1, OR13J1, OR14A2, OR14A16, OR14C36, OR14I1, OR14J1, OR14K1, OR14L1P, OR51A1P, OR51A4, OR51A7, OR51B2, OR51B4, OR51B5, OR51B6, OR51D1, OR51E1, OR51E2, OR51F1, OR51F2, OR51F5P, OR51G1, OR51G2, OR51H1, OR51I1, OR51I2, OR51L1, OR51M1, OR51Q1, OR51S1, OR51T1, OR51V1, OR52A1, OR52A4, OR52A5, OR52B2, OR52B4, OR52B6, OR52D1, OR52E2, OR52E4, OR52E5, OR52E8, OR52H1, OR52I2, OR52J3, OR52K2, OR52L2P, OR52M1, OR52N1, OR52N2, OR52N4, OR52N5, OR52P2P, OR52R1, OR52W1, OR52Z1P, OR56A1, OR56A3, OR56A4, OR56A5, OR56B1, OR56B2P, and OR56B4 (these are also referred to as “group B”).

Further examples of the G protein-coupled receptors include type I taste receptor (Tas1R), type II taste receptor (Tas2R), glucose transporter (GLUT), sodium/glucose cotransporter (SGLT), calcium sensing receptor (CasR), metabotropic glutamate receptor (mGluR), CD36, free fatty acid receptor (FFAR), and other G protein-coupled receptors. Examples of Tas1R include Tas1R1, Tas1R2, and Tas1R3. Examples of FFAR include FFAR1, FFAR2, and FFAR3. Examples of other G protein-coupled receptors include GPRC6A, GPR92, GPR120, GPR113, GPR40, GPR43, and GPR41. Each of these G protein-coupled receptors can function, for example, as a taste receptor. Tas1R1 and Tas1R3 may form, for example, a heterocomplex to function as an umami receptor. Tas1R2 and Tas1R3 may form, for example, a heterocomplex to function as a sweet taste receptor. Tas2R may function, for example, as a bitter taste receptor. GLUT and SGLT may each function, for example, as a sweet taste receptor. CasR may function, for example, as a kokumi receptor. mGluR, GPRC6A, and GPR92 may each function, for example, as an umami receptor. CD36, GPR120, and GPR113 may each function, for example, as a fat taste receptor. FFAR1 and GPR40, FFAR2 and GPR43, and FFAR3 and GPR413 may each form, for example, a heterocomplex to function as a fat taste receptor.

Examples of the ion channel-type receptors include the epithelial sodium channel (ENaC), acid-sensing ion channel (ASIC), transient receptor potential channel (TRP channel), transmembrane channel-like protein (TMC protein), voltage-dependent potassium ion channel, polycystic kidney disease 1-like 3 (PKD1L3), and polycystic kidney disease 2-like 1 (PKD2L1). Examples of the TRP channel include TRPV1t and TRPML3. Examples of the TMC protein include TMC4 and TMC6. Examples of the voltage-dependent potassium ion channel include Kv3.2. Each of these ion channel-type receptors may function, for example, as a taste receptor. Each of the ENaC, ASIC, TRP channel, TMC protein, and voltage-dependent potassium ion channel may function, for example, as a salt taste receptor. Each of the ASIC, PKD1L3, and PKD2L1 may function, for example, as a sour taste receptor.

A gene encoding a receptor is also called receptor gene.

Examples of the receptor genes and the receptors include receptor genes and receptors of various organisms. Examples of the organisms include animals such as mammals. Specific examples of the animals such as mammals include Homo sapiens (human), Mus musculus (mouse), Rattus norvegicus (rat), Canis lupus familiaris (dog), Felis catus (cat), Bos taurus (cattle), Sus scrofa (pig), Pan troglodytes (chimpanzee), Macaca fascicularis (crab-eating macaque), and Equus caballus (horse). Examples of the animals such as mammals include, in particular, humans. Nucleotide sequences of receptor genes, and amino acid sequences of receptors, of various organisms can be obtained, for example, from public databases such as NCBI and Ensembl.

The receptors may be proteins having known or natural amino acid sequences of receptors such as those described above. The receptors may also be variants of proteins having known or natural amino acid sequences of receptors such as those described above. In other words, the receptors specified by the names described above are intended to encompass, for example, proteins having known or natural amino acid sequences of the receptors specified by the names, and variants thereof. Unless otherwise specified, the expression “a protein has an amino acid sequence” means that the protein includes the amino acid sequence, and also encompasses a case where the protein is composed of the amino acid sequence. Examples of the variants include proteins having an amino acid sequence that is the same as a known or natural amino acid sequence except that one or several amino acids are substituted, deleted, inserted, and/or added at one or several positions. Specifically, the expression “one or several” may mean, for example, 1 to 50, 1 to 40, or 1 to 30, preferably 1 to 20, more preferably 1 to 10, still more preferably 1 to 5, especially preferably 1 to 3. Examples of the variants also include proteins having an amino acid sequence with an identity of not less than 50%, not less than 65%, not less than 80%, preferably not less than 90%, more preferably not less than 95%, still more preferably not less than 97%, especially preferably not less than 99% with respect to the entire known or natural amino acid sequence. A receptor specified by a biological species from which the receptor is derived is not limited to the receptor itself found in the biological species, and is intended to encompass proteins having an amino acid sequence of the receptor found in the biological species, and variants thereof. The variants may or may not be found in the biological species. Thus, for example, the term “human receptor” is not limited to the receptor itself that is found in humans, and is intended to encompass proteins having an amino acid sequence of the receptor found in humans, and variants thereof. Each receptor may be a chimeric protein of two or more receptors having different origins. Thus, the receptors specified by the names described above are intended to encompass, for example, chimeric proteins of two or more receptors specified by the name and having different origins.

The term “identity” between amino acid sequences means the identity between amino acid sequences calculated by blastp using the default scoring parameters (Matrix: BLOSUM62; Gap Costs: Existence=11, Extension=1; Compositional Adjustments: Conditional compositional score matrix adjustment).

The number of receptors is not less than 2. The number of receptors may be, for example, not less than 2, not less than 5, not less than 10, not less than 20, not less than 50, not less than 100, not less than 150, not less than 200, not less than 250, not less than 300, not less than 400, not less than 500, not less than 700, not less than 1000, or not less than 1500, and may be not more than 2000, not more than 1500, not more than 1000, not more than 700, not more than 500, not more than 400, not more than 300, not more than 250, not more than 200, not more than 150, not more than 100, not more than 50, not more than 20, not more than 10, or not more than 5. The number of receptors may also be a consistent combination of these numbers. In particular, the number of receptors may be not less than 50, not less than 100, not less than 150, not less than 200, not less than 250, or not less than 300. Specifically, for example, the number of receptors may be 2 to 5, 5 to 10, 10 to 20, 20 to 50, 50 to 100, 100 to 150, 150 to 200, 200 to 250, 250 to 300, 300 to 400, 400 to 500, 500 to 700, 700 to 1000, 1000 to 1500, or 1500 to 2000. Specifically, for example, the number of receptors may be 50 to 2000, 50 to 1000, 50 to 500, 50 to 100, 100 to 2000, 100 to 1000, 100 to 500, 300 to 2000, 300 to 1000, 300 to 500, or 300 to 400. The receptors may include, for example, some or all of the receptors exemplified above. The receptors may be composed of, for example, some or all of the receptors exemplified above. For example, not less than 50%, not less than 60%, not less than 70%, not less than 80%, not less than 90%, or not less than 95% of the total number of receptors may be selected from the receptors exemplified above. In particular, for example, not less than 50%, not less than 60%, not less than 70%, not less than 80%, not less than 90%, or not less than 95% of the total number of receptors may be selected from olfactory receptors. More particularly, for example, not less than 50%, not less than 60%, not less than 70%, not less than 80%, not less than 90%, or not less than 95% of the total number of receptors may be selected from Group A of the olfactory receptors exemplified above. More particularly, for example, not less than 50%, not less than 60%, not less than 70%, not less than 80%, not less than 90%, or not less than 95% of the total number of receptors may be selected from Group B of the olfactory receptors exemplified above. Further, the receptors may include, for example, not less than 50%, not less than 60%, not less than 70%, not less than 80%, not less than 90%, not less than 95%, or all of Group B of the olfactory receptors exemplified above. In cases where receptors form a heterocomplex to function as the heterocomplex, the heterocomplex as a whole is regarded as one type of receptor.

The method of obtaining the receptor activity profile of a substance is not limited. The receptor activity profile of a substance can be obtained, for example, by a known method of identifying the presence or absence of, or the degree of, activation or inactivation of a receptor such as an olfactory receptor or a taste receptor by the substance. Specifically, the receptor activity profile of a substance can be obtained, for example, by bringing the receptor into contact with the substance and measuring the presence or absence of, or the degree of, activation or inactivation of the receptor due to the contact with the substance. The contact between the receptor and the substance, and the measurement of the presence or absence of, or the degree of, activation or inactivation of the receptor due to the contact can be carried out, for example, with reference to a screening method for a substance that exhibits a target aroma using a response of an olfactory receptor as an index (for example, JP 2019-037197 A) or a screening method for a substance that exhibits a target taste using a response of a taste receptor as an index (for example, JP 2018-014999 A). The receptor may be used in a state where it is supported on a cell such as an animal cell. In cases where the receptor is used in a state where it is supported on the cell, the “activation or inactivation of the receptor” may mean activation or inactivation of the cell that supports the receptor. The cell supporting the receptor can be obtained, for example, by introducing a receptor gene into a cell and allowing the cell to express the gene. In cases of a receptor that functions in the form of a heterocomplex, the cell supporting the receptor can be obtained, for example, by introducing genes each encoding a subunit constituting the heterocomplex into a cell and allowing coexpression of the genes.

The cell that supports the receptor may contain a component useful or required for obtaining the receptor activity profile, such as a protein involved in signal transduction, a protein that promotes membrane expression of the receptor, or a component in accordance with the parameter to be measured. Examples of the protein involved in signal transduction include G proteins (such as Golf), G protein activators (such as Ric8B), adenylate cyclase, and calcium channels. Examples of the Golf include animal Golfs such as human Golf (GenBank accession No. NP_892023). Examples of the Ric8B include animal Ric8Bs such as rat Ric8B (GenBank accession No. NP_783188). Examples of the protein that promotes membrane expression of the receptor include RTP1s (Zhuang H and Matsunami H, J Biol Chem 282, 15284-15293 (2007)). Examples of the RTP1s include animal RTP1s such as human RTP1s (GenBank accession No. AAT70680), mouse RTP1s (GenBank accession No. ABU23737), and bat RTP1s (the amino acid sequence from the methionine residue at position 37 to the C-terminus of GenBank accession No. XP_006765914). Examples of the component in accordance with the parameter to be measured include probes such as calcium indicators and membrane potential-sensitive dyes, and reporter genes such as a luciferase gene.

Activation of a G protein-coupled receptor such as an olfactory receptor can be measured, for example, using an increase in the intracellular cAMP concentration or the intracellular calcium concentration as an index (for example, JP 2019-037197 A). For example, in HEK293T cells, activation of an olfactory receptor by an aroma component is known to cause activation of adenylate cyclase by coupling of the receptor with an intracellular G protein (such as Golf), resulting in an increased intracellular cAMP concentration (Kajiya K. et al., Molecular bases of odor discrimination: Reconstitution of olfactory receptors that recognize overlapping sets of odorants. Journal of Neuroscience, 2001, 21:6018-6025). Examples of a method of measuring the intracellular cAMP concentration include ELISA and a reporter assay. An example of the reporter assay is a luciferase assay. By the reporter assay, the intracellular cAMP concentration can be measured using a reporter gene (such as a luciferase gene) configured to be expressed in a cAMP concentration-dependent manner. An example of a method of measuring the intracellular calcium concentration is calcium imaging using a calcium indicator.

Activation of an ion channel-type receptor such as TMC protein can be measured, for example, using, as an index, influx of ions into the cell, membrane potential of the cell, inward membrane current of the cell, or an increase in the intracellular ion concentration (for example, JP 2018-014999 A). Examples of the ions include sodium ions, calcium ions, and chloride ions. Examples of a method of measuring the membrane potential include a patch clamp method and a method using a membrane potential-sensitive dye. Examples of a method of measuring the membrane current include a patch clamp method and a voltage clamp method. Examples of a method of measuring the intracellular sodium concentration include a method using a sodium indicator such as CoroNa Green Sodium Indicator (Thermo Fisher Scientific). An example of a method of measuring the intracellular calcium concentration is calcium imaging using a calcium indicator.

The contact between the receptor and the substance, and the measurement of the presence or absence of, or the degree of, activation or inactivation of the receptor due to the contact can be carried out, for example, by the following procedure.

In other words, the presence or absence of, or the degree of, activation or inactivation of the receptor by the substance can be determined using, as an index, the degree of activation of the receptor as observed upon performing the contact by bringing the receptor into contact with the substance (that is, under conditions where the receptor is brought into contact with the substance) (also referred to as “degree of activation D1”).

A system in which the contact between the receptor and the substance is carried out is also referred to as a “reaction system”. The contact between the receptor and the substance can be carried out in an appropriate liquid. In other words, the reaction system may be a liquid. A liquid in which the contact between the receptor and the substance is carried out (that is, a reaction system that is a liquid) is also referred to as “reaction liquid”. Thus, for example, the contact between the receptor and the substance can be achieved by allowing the receptor and the substance to coexist in an appropriate reaction liquid. Specifically, for example, the contact between the receptor and the substance can be achieved by dissolving, suspending, or dispersing the receptor (for example, a receptor in a form exemplified above, such as a cell having the receptor) and the substance in an appropriate liquid medium to allow their coexistence. Examples of the liquid medium include aqueous media such as water and aqueous buffer solutions. In cases where two or more components are brought together into contact with the receptor, the contact between these components and the receptor may or may not be started simultaneously. Specifically, for example, after starting the contact between a certain component and the receptor, another component may be further added to the reaction system. The reaction conditions (the conditions under which the contact between the receptor and the substance is carried out) are not particularly limited as long as the measurement of the presence or absence of, or the degree of, activation or inactivation of the receptor by the substance is possible. The reaction conditions may be set as appropriate depending on various conditions such as the mode of use of the receptor, the type of the substance, and the method of measuring activation or inactivation of the receptor. As the reaction conditions, for example, known reaction conditions for measuring interactions between substances, such as interactions between a protein and a ligand, may be used as they are or after appropriate modification of the reaction conditions. The concentration of the substance may be, for example, 0.01 nM to 500 mM, 10 nM to 100 mM, 1 μM to 10 mM, or 3 μM to 1 mM. The concentration of the substance may be, for example, 0.001 to 20% (w/w), 0.01 to 20% (w/w), 0.05 to 15% (w/w), or 0.1 to 10% (w/w). The concentration of the receptor may be, for example, 1 pg/mL to 10 mg/mL. In cases where cells having the receptor are used, the concentration of the cells having the receptor may be, for example, 10 cells/mL to 10,000,000 cells/mL. The contact between the receptor and the substance may be terminated at an appropriate time point, or may not be terminated. The contact between the receptor and the substance may usually be allowed to continue until the measurement of the presence or absence of, or the degree of, activation or inactivation of the receptor by the substance. The duration of the contact between the receptor and the substance may be, for example, not less than 0.1 seconds, not less than 0.5 seconds, not less than 1 second, not less than 5 seconds, not less than 10 seconds, not less than 30 seconds, not less than 1 minute, not less than 5 minutes, not less than 10 minutes, not less than 30 minutes, not less than 1 hour, or not less than 2 hours, and may be not more than 24 hours, not more than 12 hours, not more than 6 hours, not more than 2 hours, or not more than 1 hour. The duration of the contact may also be a consistent combination of these durations. Specifically, for example, the duration of the contact between the receptor and the substance may be 1 hour to 6 hours. The reaction system may contain, in addition to the receptor (for example, a receptor in a form exemplified above, such as a cell having the receptor) and the substance, other components as long as the measurement of the presence or absence of, or the degree of, activation or inactivation of the receptor by the substance is possible. The other components may be set as appropriate depending on various conditions such as the mode of use of the receptor, the type of the substance, and the method of measuring activation or inactivation of the receptor. Examples of the other components include salts such as calcium salts, carbon sources such as glucose, other medium components, and pH buffers.

Further, in cases where inactivation of the receptor by the substance is to be measured, the contact between the receptor and the substance may be performed, for example, in the presence of a substance (hereinafter also referred to as a “receptor activator”) that activates the receptor. The expressions “bringing a receptor into contact with a substance in the presence of a receptor activator”, “bringing a receptor into contact with a receptor activator in the presence of a substance”, and “bringing a receptor into contact with a receptor activator and a substance” may be used synonymously. The description regarding the contact between the receptor and the substance is also applicable to the contact between the receptor and the receptor activator. The contacts of the receptor activator and the substance with the receptor may or may not be started simultaneously. For example, the substance may be preliminarily mixed with the receptor activator, and the resulting mixture may be brought into contact with the receptor. Further, for example, after starting the contact between the substance and the receptor, the receptor activator may be further added to the reaction system. Alternatively, after starting the contact between the receptor activator and the receptor, the substance may be further added to the reaction system. The concentration of the receptor activator may be, for example, 0.01 nM to 500 mM, 10 nM to 100 mM, 1 μM to 10 mM, or 3 μM to 1 mM. In cases where the contact between the receptor and the substance is carried out in the presence of the receptor activator, the expression “duration of the contact between the receptor and the substance (which herein means the substance for which the receptor activity profile is to be obtained)” may be read as “duration of the contact of the receptor with the receptor activator and the substance (which herein means the substance for which the receptor activity profile is to be obtained)”.

The receptor activator is not particularly limited as long as the receptor activator can activate the receptor. The receptor activator may be composed of a single component (that is, a pure substance) or may be composed of a combination of two or more components (that is, a mixture). In cases where the receptor activator is a mixture, the number of types of the components constituting the mixture and their composition ratio are not particularly limited. The receptor activator may be a known substance, or may be a novel substance. The receptor activator may be a natural product, or may be an artificial product. The receptor activator may or may not be known to be capable of activating the receptor. The receptor activator can be screened, for example, with reference to a screening method for a substance that exhibits a target aroma using a response of an olfactory receptor as an index (for example, JP 2019-037197 A) or a screening method for a substance that exhibits a target taste using a response of a taste receptor as an index (for example, JP 2018-014999 A).

The timing of measuring the degree of activation of the receptor (degree of activation D1) upon performing the contact between the receptor and the substance is not particularly limited as long as the presence or absence of, or the degree of, activation or inactivation of the receptor by the substance can be measured. The timing of measuring the degree of activation D1 may be set as appropriate according to various conditions such as the mode of use of the receptor, the type of the substance, and the method of measuring activation or inactivation of the receptor. Specifically, the timing of measuring the degree of activation D1 may be any suitable time point from the time point when the contact between the receptor and the substance has started to the time point when the activation or inactivation of the receptor by the substance has disappeared. For example, the timing of measuring the degree of activation D1 may be a time point when the degree of activation or inactivation of the receptor by the substance is maximum. Further, the timing of measuring the degree of activation D1 may be, for example, not less than 0.1 seconds, not less than 0.5 seconds, not less than 1 second, not less than 5 seconds, not less than 10 seconds, not less than 30 seconds, not less than 1 minute, not less than 5 minutes, not less than 10 minutes, not less than 30 minutes, not less than 1 hour, or not less than 2 hours after the time when the contact between the receptor and the substance has started, and may be not more than 24 hours, not more than 12 hours, not more than 6 hours, not more than 2 hours, or not more than 1 hour after the time when the contact between the receptor and the substance has started. The timing of measuring the degree of activation D1 may also be a consistent combination of these timings. Specifically, the timing of measuring the degree of activation D1 may be, for example, from not less than 1 hour to not more than 6 hours after the time when the contact between the receptor and the substance has started. In cases where the contact between the receptor and the substance is carried out in the presence of the receptor activator, the expression “the time point when the contact between the receptor and the substance (which herein means the substance for which the receptor activity profile is to be obtained) has started” may be read as “the time point when the contact of the receptor with the receptor activator and the substance (which herein means the substance for which the receptor activity profile is to be obtained) has started.”

Specifically, the presence or absence of, or the degree of, activation or inactivation of the receptor by the substance can be determined by comparing the degree of activation D1 with the degree of activation of the receptor under a control condition (also referred to as “degree of activation D2”). The control condition may be, for example, a condition in which the receptor is not brought into contact with the substance. In cases where the contact between the receptor and the substance (which herein means the substance for which the receptor activity profile is to be obtained) is carried out in the presence of the receptor activator, the control condition may be, for example, a condition in which the receptor is brought into contact with the receptor activator but not brought into contact with the substance (that is, a condition in which the receptor is brought into contact with the receptor activator in the absence of the substance).

The degrees of activation D1 and D2 can both be obtained and used as data reflecting a parameter that serves as an index of activation or inactivation of the receptor. Examples of the parameter that serves as an index of activation or inactivation of the receptor include the amount of intracellular calcium and the amount of intracellular cAMP. In cases of a luciferase assay, examples of the data reflecting the amount of intracellular cAMP include luminescence intensity. The data reflecting the parameter that serves as an index of activation or inactivation of the receptor can be used as it is, or after being subjected to processing as appropriate. Examples of the processing include logarithmic transformation, correction, and standardization. The processing such as logarithmic transformation, correction, or standardization can be carried out, for example, according to the procedure described in the Examples.

In cases where the degree of activation D1 is high, the receptor may be judged to have been activated by the substance. For example, in cases where the ratio of the degree of activation D1 to the degree of activation D2 (that is, D1/D2) falls within a predetermined range, the receptor may be judged to have been activated by the substance. The predetermined range in which the receptor is judged to have been activated by the substance may be set as appropriate according to various conditions such as the type of the receptor and the types of data representing the degrees of activation D1 and D2. The predetermined range in which the receptor is judged to have been activated by the substance may be, for example, not less than 1.5, not less than 2, not less than 3, not less than 5, not less than 10, not less than 20, not less than 50, or not less than 100. The ratio of the degree of activation D1 to the degree of activation D2 may be, for example, a normalized response value calculated by the procedure described in JP 2019-037197 A.

In cases where the degree of activation D1 is low, the receptor may be judged to have been inactivated by the substance. For example, in cases where the ratio of the degree of activation D1 to the degree of activation D2 (that is, D1/D2) falls within a predetermined range, the receptor may be judged to have been activated by the substance. The predetermined range in which the receptor is judged to have been inactivated by the substance may be set as appropriate according to various conditions such as the type of the receptor and the types of data representing the degrees of activation D1 and D2. The predetermined range in which the receptor is judged to have been inactivated by the substance may be, for example, not more than 0.8, not more than 0.7, not more than 0.6, not more than 0.5, not more than 0.4, not more than 0.3, not more than 0.2, or not more than 0.1. The ratio of the degree of activation D1 to the degree of activation D2 may be, for example, a normalized response value calculated by the procedure described in JP 2019-037197 A.

Further, the degree of activation or inactivation of the receptor by the substance can also be determined using, as an index, a comparison result between the degree of activation D1 and the degree of activation D2. For example, the ratio of the degree of activation D1 to the degree of activation D2 (that is, D1/D2) can be regarded as the degree of activation or inactivation of the receptor by the substance. The ratio of the degree of activation D1 to the degree of activation D2 may be, for example, a “normalized response value” calculated by the procedure described in JP 2019-037197 A. Further, for example, a value obtained by subtracting a logarithmic value of the degree of activation D1 from a logarithmic value of the degree of activation D2, carrying out correction as appropriate, and then performing standardization among receptors can be regarded as the degree of activation or inactivation of the receptor by the substance. Such a standardized value may be, for example, a “response value” described in the Examples.

The receptor activity profile may be processed by, for example, nonlinear transformation. For example, data representing the degree of activation or inactivation of the receptor by the substance may be subjected to processing such as nonlinear transformation and then used as a receptor activity profile. The mode of processing may be set as appropriate according to various conditions such as the mode of data to be processed and the purpose of processing. For example, data to be emphasized that are included in the target profile may be relatively enhanced. Examples of the data to be emphasized include data for receptors to be emphasized. Examples of the receptors to be emphasized include receptors that respond strongly to a target article that exhibits a target sensory quality, and receptors that respond weakly to a target article that exhibits a target sensory quality. By relatively enhancing data for a certain receptor and subjecting the resulting data to the identification step, target substances and their combination ratio that provide a composite profile that approximates the target profile may be identified such that priority is placed on the receptor activity profile portion for the certain receptor. Thus, for example, by selecting, as the receptors to be emphasized, receptors that respond strongly to a target article that exhibits a target sensory quality, the influence of the receptors may be evaluated more strongly. Further, for example, by selecting, as the receptors to be emphasized, receptors that respond weakly to a target article that exhibits a target sensory quality, the influence of the receptors may be evaluated without being overlooked.

The means of carrying out the identification step is not particularly limited as long as the target substances and their combination ratio can be identified with a desired accuracy.

The identification step can be carried out, for example, by an analytical technique that seeks a solution to an inverse problem (in particular, a solution to an inverse problem of an underdetermined system), using the target profile as an objective variable, and using the receptor activity profile, for the receptors, of each substance constituting the test substance group as an explanatory variable. The analytical technique that seeks a solution to an inverse problem of an underdetermined system is, for example, sparse modeling. Since the prediction method of the present invention predicts a formulation of substances, the sparse modeling may be carried out such that coefficients of the explanatory variables are positive (that is, under a nonnegative constraint condition). Thus, the sparse modeling may be nonnegative constraint sparse modeling.

In the sparse modeling, regularization may be carried out. Examples of the regularization include L1 regularization, L2 regularization, and L0 regularization. Examples of the regularization include, in particular, L1 regularization. By the regularization such as L1 regularization, for example, variables may be reduced. The strength of the regularization may be appropriately set depending on various conditions such as the number of test substances and the number of target substances. The strength of the regularization may be set using a regularization parameter λ. The larger the regularization parameter λ, the larger the number of regression coefficients estimated as zero (or almost zero) may be, that is, the smaller the number of target substances selected may be. Further, the larger the regularization parameter λ, the lower the risk of overfitting may be, whereas the higher the risk of underfitting may be. Further, the modeling may be executed a plurality of times with different strengths of regularization (specifically, with different values of the regularization parameter λ). The strength of the regularization may be set either commonly or independently among explanatory variables (that is, among test substances). For example, by using the regularization parameter λ, the overall strength of the regularization may be set (that is, the strength of the regularization may be set commonly among all explanatory variables). Furthermore, by using the regularization parameter λ together with a penalty factor that is set for each explanatory variable, the strength of the regularization may be set independently among the explanatory variables. By setting the strength of the regularization independently among the explanatory variables (that is, among the test substances), the tendency of each test substance to be selected as a target substance can be controlled. For example, some or all of the test substances may be preliminarily selected as target substances. Specifically, for example, by setting the penalty factor of a test substance to zero, the test substance can be preliminarily selected as a target substance. Further, for example, some of the test substances may be preliminarily excluded from the target substances. Specifically, for example, by setting the penalty factor of a test substance to infinity, the test substance can be preliminarily excluded from the target substances.

Examples of an algorithm for the regularization or sparse modeling including the regularization include the LASSO regression model, the Ridge regression model, and the Elastic Net regression model. According to the LASSO regression model, L1 regularization may be carried out. According to the Ridge regression model, L2 regularization may be carried out. According to the Elastic Net regression model, L1 regularization and L2 regularization may be carried out in combination. Examples of the algorithm for the regularization or for sparse modeling including the regularization include, in particular, the LASSO regression model.

By carrying out an analytical technique such as sparse modeling in this manner, candidates for the target substances and their combination ratios can be obtained. Among such candidates, those that provide a composite profile that approximates the target profile may be employed as a solution. For example, among such candidates, those that provide a composite profile that approximates the target profile, and whose number of target substances is within a predetermined range, may be employed as a solution. As the solution, one solution may be employed, or two or more solutions may be employed.

A solution that provides a composite profile that approximates the target profile may be selected, for example, based on an error between the composite profile and the target profile. The error between the composite profile and the target profile may be calculated, for example, by any evaluation function that provides a difference between these profiles as a nonnegative numerical value. Examples of the evaluation function include the mean absolute error (MAE), the mean squared error (MSE), the root mean squared error (RMSE), the mean squared logarithmic error (MSLE), the root mean squared logarithmic error (RMSLE), the mean absolute percentage error (MAPE), and the root mean squared percentage error (RMSPE). Examples of the evaluation function include, in particular, squared errors such as MSE, RMSE, MSLE, RMSLE, and RMSPE. More particularly, the evaluation function is, for example, RMSE. Any of the evaluation functions exemplified above may be used, for example, to calculate a weighted error. The degree of error at which the composite profile is determined to approximate the target profile may be appropriately set, for example, depending on various conditions such as the type of evaluation function and the number of target substances. The expression “the composite profile approximates the target profile” may mean, for example, that an error calculated by an evaluation function exemplified above is not more than a predetermined value. Specifically, the expression “the composite profile approximates the target profile” may mean, for example, that the error between the composite profile and the target profile is not more than 0.75, not more than 0.74, not more than 0.73, not more than 0.72, not more than 0.71, not more than 0.7, not more than 0.69, not more than 0.68, not more than 0.67, not more than 0.66, not more than 0.65, not more than 0.64, not more than 0.63, not more than 0.62, not more than 0.61, or not more than 0.6 in terms of a weighted RMSE. In particular, the error between the composite profile and the target profile may be, for example, not more than 0.7 in terms of a weighted RMSE.

In this manner, the target substances and their combination ratio can be identified, and hence a target formulation can be predicted. The formulation of target substances at the combination ratio identified in the identification step may be regarded as the target formulation, and hence may be predicted as the target formulation. In other words, the target substances and their combination ratio identified in the identification step may be regarded as the substances to be blended in the target formulation and their blending ratio, and hence may be predicted as the substances to be blended in the target formulation and their blending ratio.

The method of the present invention may further comprise a step of evaluating the sensory quality of the predicted target formulation. In other words, by evaluating the sensory quality of the predicted target formulation, whether the predicted target formulation actually exhibits the target sensory quality can be confirmed. There is no particular limitation on the technique for evaluating the sensory quality of the predicted target formulation. For example, the sensory quality of the predicted target formulation can be evaluated by a known technique for evaluating a sensory quality such as aroma or taste of a substance. Examples of such a technique include sensory evaluation (evaluation by a sensory test). Specifically, for example, target substances may be blended based on the predicted target formulation, and the sensory quality of the resulting formulation may be evaluated. To the blending of the target substances for evaluating the sensory quality, the description on blending of target substances in the reconstruction method of the present invention described later is applicable.

### <1-3> Reconstruction Method of the Present Invention

The target sensory quality can be reconstructed based on the predicted target formulation. In other words, the reconstruction method of the present invention may be a method of reconstructing a target sensory quality, the method comprising the steps of: predicting a formulation that exhibits a target sensory quality by the prediction method of the present invention; and blending the target substances based on the formulation.

The blending ratio of the target substances is not particularly limited as long as the target sensory quality can be reconstructed. For example, the target substances may be blended at a blending ratio that is substantially identical to the combination ratio identified in the identification step. The expression “a blending ratio that is substantially identical to the combination ratio identified in the identification step” means, for example, that the blending ratio of each target substance may be not less than 0.8 times, not less than 0.85 times, not less than 0.88 times, not less than 0.9 times, not less than 0.91 times, not less than 0.92 times, not less than 0.93 times, not less than 0.94 times, not less than 0.95 times, not less than 0.96 times, not less than 0.97 times, not less than 0.98 times, not less than 0.99 times, not less than 1 times, not less than 1.01 times, not less than 1.02 times, not less than 1.03 times, not less than 1.04 times, not less than 1.05 times, not less than 1.06 times, not less than 1.07 times, not less than 1.08 times, not less than 1.09 times, not less than 1.1 times, not less than 1.12 times, or not less than 1.15 times the combination ratio identified in the identification step, and may be not more than 1.2 times, not more than 1.15 times, not more than 1.12 times, not more than 1.1 times, not more than 1.09 times, not more than 1.08 times, not more than 1.07 times, not more than 1.06 times, not more than 1.05 times, not more than 1.04 times, not more than 1.03 times, not more than 1.02 times, not more than 1.01 times, not more than 1 times, not more than 0.99 times, not more than 0.98 times, not more than 0.97 times, not more than 0.96 times, not more than 0.95 times, not more than 0.94 times, not more than 0.93 times, not more than 0.92 times, not more than 0.91 times, not more than 0.9 times, not more than 0.88 times, or not more than 0.85 times the combination ratio identified in the identification step. The blending ratio may also be a consistent combination of these ratios. Specifically, the expression “a blending ratio that is substantially identical to the combination ratio identified in the identification step” means, for example, that the blending ratio of each target substance may be 0.8 to 0.85 times, 0.85 to 0.88 times, 0.88 to 0.9 times, 0.9 to 0.91 times, 0.91 to 0.92 times, 0.92 to 0.93 times, 0.93 to 0.94 times, 0.94 to 0.95 times, 0.95 to 0.96 times, 0.96 to 0.97 times, 0.97 to 0.98 times, 0.98 to 0.99 times, 0.99 to 1 times, 1 to 1.01 times, 1.01 to 1.02 times, 1.02 to 1.03 times, 1.03 to 1.04 times, 1.04 to 1.05 times, 1.05 to 1.06 times, 1.06 to 1.07 times, 1.07 to 1.08 times, 1.08 to 1.09 times, 1.09 to 1.1 times, 1.1 to 1.12 times, 1.12 to 1.15 times, or 1.15 to 1.2 times the combination ratio identified in the identification step. Specifically, the expression “a blending ratio that is substantially identical to the combination ratio identified in the identification step” means, for example, that the blending ratio of each target substance may be 0.85 to 1.15 times, 0.9 to 1.1 times, 0.95 to 1.05 times, or 0.98 to 1.02 times the combination ratio identified in the identification step.

### <1-4> Reconstruction Method for Target Set of Interest

In general, in order to reconstruct sensory qualities for various targets (a target set) using a single apparatus, a large number of target substances need to be loaded in the apparatus. In view of this, a set of synthetic elements may be optimized such that a plurality of target sensory qualities can be reconstructed by combination of a limited number of synthetic elements. The term “synthetic elements” refers to one or more test substances (target substances) selected from a test substance group. In other words, a synthetic element may include two or more test substances blended at a predetermined ratio. A set of a predetermined number of synthetic elements for reconstruction of sensory qualities for a plurality of targets is referred to as a minimum set.

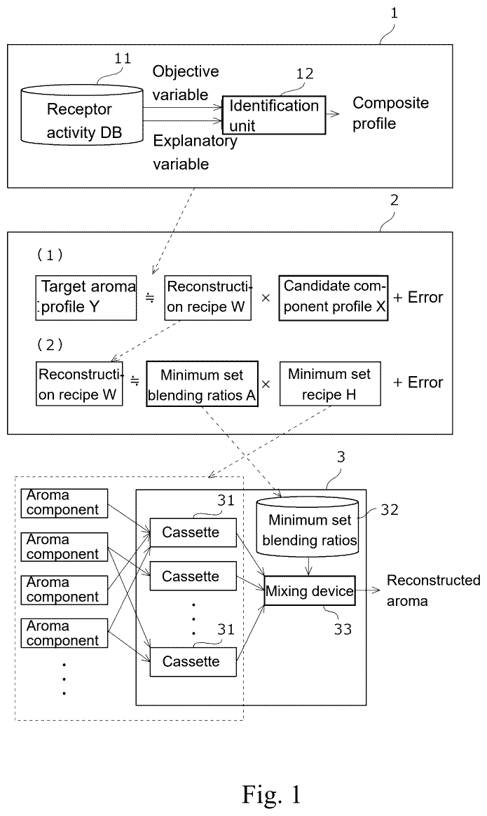

FIG. 1 is a diagram for illustrating reconstruction of a target set of interest. In FIG. 1, aroma, which is an example of a sensory quality, is reconstructed. Unlike, for example, color, for which three primary colors have been defined, aroma has no basic elements that have been defined to allow reconstruction of any aroma by blending them. Therefore, it is preferred to optimize the minimum set according to the target set of interest. The system illustrated in FIG. 1 comprises: a prediction device 1 that executes the prediction method of the present invention; an optimization device 2 that creates a minimum set of synthetic elements; and a reconstruction device 3 that is provided with a plurality of cassettes (cartridges) storing the synthetic elements, that mixes the synthetic elements, and that outputs a reconstructed aroma.

The prediction device 1 is a computer that performs the identification step described in <1-2>. The prediction device 1 comprises a receptor activity database (DB) 11 and an identification unit 12. The receptor activity DB 11 is a database stored in a storage device included in the computer, and receptor activity profiles are registered in the database such that they correspond to the respective test substances constituting a test substance group. In addition, a processor included in the computer executes a program according to the embodiment, to function as the identification unit 12 that performs the identification step. The identification unit 12 uses a receptor activity profile of a desired target as an objective variable and uses other test substances as explanatory variables to identify a composite profile.

The reconstruction device 3 is an apparatus that includes a predetermined number of cassettes 31, and reconstructs a plurality of target sensory qualities of interest. The device includes a computer known in the art. Each cassette 31 is a container storing one synthetic element, and, for example, a plurality of cassettes 31 are detachably mounted on the reconstruction device 3. The number of cassettes 31 (synthetic elements) is not particularly limited. For example, about 2 to 24 cassettes may be employed. Minimum set blending ratios 32 are information stored in a storage device of the computer. The minimum set blending ratios 32 include blending ratios of synthetic elements that provide composite profiles each of which corresponds to and approximates each of the plurality of targets of interest. A mixing device 33 includes a processor of the computer. The mixing device 33 extracts synthetic elements from the cassettes 31 at the ratios indicated by the minimum set blending ratios 32, mixes the extracted synthetic elements, and outputs the resulting mixture.

The optimization device 2 is the so-called computer. The optimization device 2 determines the synthetic elements to be stored in the cassettes 31 and recipes of the respective synthetic elements according to the plurality of targets of interest. The optimization device 2 may be a device integrated with at least one of the prediction device 1 and the reconstruction device 3.

In (1) of FIG. 1, the optimization device 2 identifies a reconstruction recipe W for reconstructing a receptor activity profile Y of a target aroma. The receptor activity profile Y of the target aroma can be expressed as an m-row by d-column matrix that represents d-dimensional receptor activity profiles corresponding to each of m types of targets of interest. The reconstruction recipe W can be expressed as an m-row by k-column matrix that represents k types of candidate components corresponding to each of the m types of targets of interest. The k types of candidate components are, for example, a union of the target substances identified in the identification step. A receptor activity profile X of the candidate components can be expressed as a k-row by d-column matrix that represents d-dimensional receptor activity profiles corresponding to each of the k types of candidate components. For each target aroma, the prediction device 1 performs the identification step to allow determination of the reconstruction recipe W. For the identification step (1), a relatively large number of target substances are preferably set (to provide a margin) before the execution of the step. It should be noted that each composite profile that approximates a target profile, obtained by the identification step, includes an error for the target aroma.

In (2) of FIG. 1, the optimization device 2 calculates a minimum set for reconstructing the target aroma set based on the reconstruction recipe W. Minimum set blending ratios A (basis matrix A) can be expressed as an m-row by n-column matrix that represents n types of synthetic elements corresponding to each of the m types of targets of interest. A minimum set recipe H (coefficient matrix H) can be expressed as an n-row by k-column matrix that represents blending ratios of the k types of candidate components for each of the n types of synthetic elements. The reconstruction recipe W can be decomposed, by nonnegative matrix factorization (NMF), into the minimum set recipe H and the minimum set combination ratios A. Based on the minimum set recipe H determined in this manner, each of the cassettes 31 is charged with a synthetic element. The minimum set blending ratios A are stored as the minimum set blending ratios 32 of the reconstruction device 3. By performing the identification step in (1) with a margin, candidate components available for creating the minimum set recipe H can be increased, and errors in the receptor activity profiles can be reduced between the reconstructed aroma finally output by the reconstruction device 3 and the target aroma to be reproduced.

In this manner, the cassettes 31 (synthetic elements) for reconstructing a desired target set of interest can be optimized. The processes (1) and (2) in FIG. 1 may be executed in the reverse order. Further, the number of candidate components (k), the number of targets (m), and the number of synthetic elements (n) preferably satisfy the inequality “k>m>n”. Thus, the reconstruction device 3 is preferably capable of employing a relatively small number (n) of synthetic elements (cassettes 31) to reproduce a larger number of types (m types) of targets. The value of n may be arbitrarily set by a user when the NMF is performed. The value of n is determined as appropriate, for example, based on the scale of the reconstruction device 3, and the errors in the receptor activity profiles between the reconstructed aroma and the target aroma to be reproduced. It should be noted that the number of candidate components (k) is generally larger than the number of targets (m).

### <2> Program of the Present Invention

The present invention provides a program that causes a computer to execute the steps included in the method of the present invention. This program is also referred to as “the program of the present invention.”

Thus, the computer may execute the steps included in the method of the present invention. The computer may execute some or all of the steps included in the method of the present invention. For example, the computer may execute the step of obtaining a target profile, the step of obtaining receptor activity profiles of test substances, and/or the identification step. In particular, for example, the computer may execute the identification step. Specifically, for example, based on a target profile input in the computer, the program of the present invention may cause the computer to execute the identification step, and a result of identification of target substances and their combination ratio (that is, a result of prediction of the target formulation) may be output. The receptor activity profiles of the test substances used in the identification step may be, for example, preliminarily stored in the computer, or may be input into the computer at the time when the identification step is carried out.

The program of the present invention may be stored in, and provided as, a computer-readable storage medium. The term “computer-readable storage medium” refers to a storage medium in which information, such as data or a program, is stored by electrical, magnetic, optical, mechanical, or chemical action, and from which the stored information can be read by a computer. Examples of such a storage medium include a floppy (registered trademark) disk, a magneto-optical disk, CD-ROM, CD-R/W, DVD-ROM, DVD-R/W, DVD-RAM, DAT, an 8-mm tape, a memory card, a hard disk, a read only memory (ROM), and SSD. Further, regarding the program of the present invention, steps to be executed by a computer may be stored as separate programs.

### EXAMPLES

The present invention is described below in more detail with reference to non-limiting Examples.

### <1> Preparation of Human Olfactory Receptor-Expressing Cells

### <1-1> Preparation of Expression Vectors for Human Olfactory Receptors

As olfactory receptors, 352 types (OR1A1, OR1A2, OR1B1, OR1C1, OR1D2, OR1D5, OR1E1, OR1F1, OR1F12, OR1G1, OR1I1, OR1J1, OR1J2, OR1J4, OR1K1, OR1L1, OR1L3, OR1L4, OR1L8, OR1M1, OR1N1, OR1N2, OR1Q1, OR1R1P, OR1S1, OR2A1, OR2A2, OR2A4, OR2A5, OR2A12, OR2A14, OR2A25, OR2AE1, OR2AG1, OR2AG2, OR2AJ1P, OR2AK2, OR2AP1, OR2AT4, OR2B2, OR2B3, OR2B6, OR2B11, OR2C1, OR2C3, OR2D2, OR2D3, OR2F1, OR2G2, OR2G3, OR2G6, OR2H1, OR2H2, OR2J2, OR2J3, OR2K2, OR2L2, OR2L8, OR2L13, OR2M2, OR2M4, OR2M7, OR2S2, OR2T1, OR2T2, OR2T5, OR2T6, OR2T8, OR2T10, OR2T11, OR2T27, OR2T34, OR2V2, OR2W1, OR2W3, OR2Y1, OR2Z1, OR3A1, OR3A2, OR3A3, OR3A4, OR4A5, OR4A15, OR4A16, OR4A47, OR4B1, OR4C3, OR4C5, OR4C6, OR4C11, OR4C12, OR4C13, OR4C15, OR4C16, OR4C46, OR4D1, OR4D2, OR4D5, OR4D6, OR4D9, OR4D10, OR4D11, OR4E2, OR4F3, OR4F5, OR4F6, OR4F14P, OR4F15, OR4G11P, OR4H12P, OR4K1, OR4K2, OR4K5, OR4K13, OR4K14, OR4K15, OR4K17, OR4L1, OR4M1, OR4N2, OR4N4, OR4N5, OR4P4, OR4Q3, OR4S1, OR4S2, OR4X1, OR4X2, OR5A1, OR5A2, OR5AC2, OR5AK2, OR5AK3P, OR5AN1, OR5AP2, OR5AR1, OR5AS1, OR5AU1, OR5B2, OR5B3, OR5B12, OR5B17, OR5B21, OR5C1, OR5D13, OR5D14, OR5D16, OR5D18, OR5F1, OR5H1, OR5H2, OR5H6, OR5H14, OR5I1, OR5J2, OR5K1, OR5K3, OR5K4, OR5L2, OR5M3, OR5M8, OR5M9, OR5M10, OR5M11, OR5P3, OR5R1, OR5T1, OR5T2, OR5T3, OR5V1, OR5W2, OR6A2, OR6B1, OR6B2, OR6C1, OR6C2, OR6C3, OR6C4, OR6C6, OR6C65, OR6C66P, OR6C68, OR6C70, OR6C74, OR6C75, OR6C76, OR6F1, OR6J1, OR6K2, OR6K3, OR6K6, OR6M1, OR6N1, OR6N2, OR6P1, OR6Q1, OR6S1, OR6T1, OR6V1, OR6X1, OR6Y1, OR7A3P, OR7A5, OR7A10, OR7A17, OR7C1, OR7C2, OR7D2, OR7D4, OR7E24, OR7G1, OR7G2, OR7G3, OR8A1, OR8B3, OR8B4, OR8B8, OR8B12, OR8D1, OR8D2, OR8D4, OR8G2, OR8G5, OR8H3, OR8I2, OR8J1, OR8J3, OR8K1, OR8K3, OR8K5, OR8S1, OR8U1, OR9A4, OR9G1, OR9G4, OR9I1, OR9K2, OR9Q1, OR9Q2, OR10A3, OR10A4, OR10A5, OR10A6, OR10A7, OR10AD1, OR10AG1, OR10C1, OR10D3, OR10D4P, OR10G2, OR10G3, OR10G4, OR10G6, OR10G7, OR10G9, OR10H2, OR10H4, OR10J1, OR10J3, OR10J5, OR10K1, OR10K2, OR10P1, OR10Q1, OR10R2, OR10S1, OR10T2, OR10V1, OR10W1, OR10X1, OR10Z1, OR11A1, OR11G2, OR11H4, OR11H6, OR11H12, OR11L1, OR12D2, OR12D3, OR13A1, OR13C2, OR13C3, OR13C4, OR13C8, OR13D1, OR13F1, OR13G1, OR13H1, OR13J1, OR14A2, OR14A16, OR14C36, OR14I1, OR14J1, OR14K1, OR14L1P, OR51A1P, OR51A4, OR51A7, OR51B2, OR51B4, OR51B5, OR51B6, OR51D1, OR51E1, OR51E2, OR51F1, OR51F2, OR51F5P, OR51G1, OR51G2, OR51H1, OR51I1, OR51I2, OR51L1, OR51M1, OR51Q1, OR51S1, OR51T1, OR51V1, OR52A1, OR52A4, OR52A5, OR52B2, OR52B4, OR52B6, OR52D1, OR52E2, OR52E4, OR52E5, OR52E8, OR52H1, OR52I2, OR52J3, OR52K2, OR52L2P, OR52M1, OR52N1, OR52N2, OR52N4, OR52N5, OR52P2P, OR52R1, OR52W1, OR52Z1P, OR56A1, OR56A3, OR56A4, OR56A5, OR56B1, OR56B2P, and OR56B4) of human olfactory receptors were employed.

From the TrueClone CDNA Clone Collection (OriGene), 352 types of human olfactory receptor genes were purchased. Using primers designed on the basis of sequence information deposited in GenBank, a PCR method was carried out using the purchased human olfactory receptor genes as templates, to amplify fragments for subcloning of the 352 types of human olfactory receptor genes. The amplified fragment for subcloning of each gene was subcloned downstream of the Rho tag sequence of a Rho-pME18S vector (K. Kajiya et al., Journal of Neuroscience 15 Aug. 2001, 21 (16) 6018-6025) utilizing EcoRI and Xhol sites, to obtain 352 types of expression vectors for the human olfactory receptors.

### <1-2> Preparation of Olfactory Receptor-Expressing Cells

HEK293T cells expressing each of the 352 types of olfactory receptors were prepared by the following procedure. The gene mixed liquid shown in Table 1 and the transfection reagent mixed liquid shown in Table 2 were prepared, and left to stand at room temperature for 5 minutes. pcDNA3.1-microbat RTP1s is an expression vector for microbat RTP1s, pcDNA3.1-human Golf is an expression vector for human Golf, and pcDNA3.1-rat Ric8B is an expression vector for rat Ric8B (JP 2019-037197 A). The gene mixed liquid and the transfection reagent mixed liquid were mixed together, and the resulting mixture was aliquoted in 12.5-μL volumes into wells of a poly-D-lysine coat 384-well plate. The plate was then left to stand in a clean bench for 15 minutes. From HEK293T cells that had been seeded in a 10-cm Petri dish (2.5×10⁶ cells/10-cm Petri dish) on the previous day, a cell suspension was prepared at 1.2×10⁵ cells/mL. The cell suspension was seeded, in 25-μL volumes, into the wells of the 384-well plate, and then the cells were cultured overnight in an incubator maintained at 37° C. and 5% CO₂. In this manner, the expression vectors shown in Table 1 were transfected to obtain 352 types of HEK293T cell cultures that appropriately express the genes encoded by the expression vectors.

TABLE 1

| | | |
|---|---|---|
| Opti-MEM (Gibco) | 6.2 | μL |
| Expression vector for human olfactory receptor | 0.0125 | μg |
| pGL4.29[luc2P/CRE/Hygro] Vector (Promega) | 0.0025 | μg |
| pGL4.74[hRluc/TK] Vector (Promega) | 0.00125 | μg |
| pcDNA3.1-microbat RTP1s | 0.0025 | μg |
| pcDNA3.1-human Golf | 0.00125 | μg |
| pcDNA3.1-rat Ric8B | 0.00125 | μg |

TABLE 2

| | | |
|---|---|---|
| Opti-MEM (Gibco) | 6.2 | μL |
| Lipofectamine 2000 (Invitrogen) | 0.0425 | μL |

### <2> Preparation of a Human Olfactory Receptor Activity Database

### <2-1> Luciferase Assay

Responses of the olfactory receptors to test substances were measured using the olfactory receptor-expressing cells.

The 352 types of olfactory receptors expressed in the HEK293T cells couple with Golf to activate adenylate cyclase, thereby increasing the amount of intracellular cAMP. In the present Example, the measurement of the responses of the olfactory receptors to the test substances was carried out using a luciferase reporter gene assay that monitors an increase in the amount of intracellular cAMP as an increase in the luminescence value derived from firefly luciferase. The “luciferase reporter gene assay” is also referred to as a “luciferase assay”. The firefly luciferase is expressed in a manner dependent on the amount of intracellular cAMP, from a firefly luciferase gene carried by the pGL4.29[luc2P/CRE/Hygro] Vector. In addition, the luminescence value derived from Renilla luciferase was used as an internal standard for correcting errors in the gene transfer efficiency and the cell number among the wells. The Renilla luciferase is constitutively expressed under the control of a TK promoter from the Renilla luciferase gene carried by the pGL4.74[hRluc/TK] Vector.

As test substances, 3103 types of substances including, for example, substances described in The Good Scents Company (http://www.thegoodscentscompany.com/) were selected. The culture medium was removed from the 352 types of cultures obtained in <1-2> above, and 15 μL of each of 3103 types of test substance solutions was added to each of the cultures, to obtain 352×3103 types of reaction liquids. Each test substance solution was prepared by dissolving each test substance in CD293 (Life Technologies, Inc.) The test substance concentration in the test substance solution was set to 300 μM in principle. However, for each test substance that exhibited cytotoxicity at 300 μM, the test substance concentration in the test substance solution was set to 3 μM, 10 μM, 30 μM, or 100 μM. For a very small number of test substances, the test substance concentration in the test substance solution was set to 1000 μM. In cases where the test substance was a mixture, the concentration was set to 0.1%, 0.3%, 0.5%, 1%, 3%, 5%, 10%, 50%, or 100%. The reaction liquids were placed in an incubator maintained at 37° C. and 5% CO₂, and the cells were cultured for 4 hours to allow sufficient expression of the firefly luciferase gene in the cells. The luminescence value derived from the intracellular firefly luciferase was measured to determine the “Luc value”. The luminescence value derived from the intracellular Renilla luciferase was also measured to determine the “hRLuc value”. The luminescence value derived from each luciferase was measured using the Dual-Glo luciferase assay system (Promega) in accordance with the operating manual of the product.

### <2-2> Correction and Normalization of Reporter Signals

Since signals obtained in the luciferase assay may be biased due to various factors such as experimental conditions, direct use of the signals for exhaustive analysis or statistical modeling may only result in detection of such a bias, so that a desired result may not be obtained. In view of this, correction and normalization of reporter signals were carried out by the methods described below to obtain a profile composed of activities of the 352 types of olfactory receptors for each test substance. Specifically, the correction and normalization were carried out by the following: elimination of distortion of the distribution of reporter signals using logarithmic transformation, correction of the gene transfer efficiency using an internal standard, correction of inter-experiment differences in reporter signals using an unstimulated condition, correction of basal activity specific to each olfactory receptor, correction of olfactory receptor-independent activity, and normalization of activity values using the mean value and the standard deviation of all data.

### <3> Reconstruction of a Target Aroma Using Olfactory Receptor Activity Profiles as an Index

### <3-1> Aroma Reconstruction Targeting a Single Aroma Component

From a group of 3103 types of test substances, the two substances shown in Table 3 were selected and used as target aromas (that is, aromas to be reconstructed). Olfactory receptor activity profiles of the targets in an olfactory receptor activity database were set as target profiles.

TABLE 3

| Target (single component) | CAS number |
|---|---|
| Syringol | 91-10-1 |
| trans,trans-2,4-Decadienal | 25152-84-5 |

For each target, the 3102 types of substances obtained by excluding the target from the olfactory receptor activity database were used as a test substance group, and olfactory receptor activity profiles of these test substances were set as a test substance group profile.

Using the target profile as an objective variable (352 points×1 variable), and the test substance group profile as explanatory variables (352 points×3102 variables), nonnegative constraint linear sparse modeling was performed to obtain a composite profile that approximates the target profile, and coefficients of the variables (the combination ratios of the receptor activity profiles of two or more target substances that provide the composite profile). Specifically, a LASSO (least absolute shrinkage and selection operator) regression model was used. The intercept of the model was set to 0. Using a parameter λ that defines the strength of regularization, optimization was performed such that the number of variables having a coefficient greater than 0 (the number of two or more target substances that provide the composite profile) was not more than 4. The mixing ratio of the target substances was determined according to the ratio of the coefficients of the model.

The target substances were mixed according to the calculated mixing ratio to prepare each reconstructed product. For sensory evaluation, each of the target and the reconstructed product was diluted to a concentration at which the aroma could be smelled without difficulty. Table 4 shows the compositions of the evaluation samples used for the sensory evaluation.

TABLE 4
Compositions of evaluation samples used for sensory evaluation

| | | | | Evaluation |
|---|---|---|---|---|
| | | Test | CAS | concen- |
| Test | Sample | substance | number | tration |
| 1 | Target | Syringol | 91-10-1 | 1.54% |
| | Reconstructed | Guaiacol | 90-05-1 | 0.53% |
| | product | 4-Ethylguaiacol | 2785-89-9 | 0.41% |
| | | 4-Vinylguaiacol | 7786-61-0 | 0.30% |
| | | 2-Methoxy-5- | 1195-09-1 | 0.14% |
| | | methylphenol | | |
| 2 | Target | trans,trans-2,4- | 25152-84-5 | 1.52% |
| | | Decadienal | | |
| | Reconstructed | trans,trans-2,4- | 5910-87-2 | 1.22% |
| | product | Nonadienal | | |
| | | 3- | 620-22-4 | 0.14% |
| | | Methylbenzonitrile | | |

The sensory evaluation was carried out in a blind manner (n=4). A target aroma was presented to each panelist as a reference sample. Subsequently, as an evaluation sample, the target aroma, the aroma of the reconstructed product, or a constituent component thereof was presented once each in a random order. Each panelist evaluated how similar the quality of aroma of each evaluation sample was to the quality of aroma of the reference sample, using a 5-point scale (1 point: not similar at all; 2 points: rather dissimilar; 3 points: neither similar nor dissimilar; 4 points: relatively similar; 5 points: very similar). For each evaluation sample, the median of the scores given by all panelists was calculated to determine the “sensory similarity” to the target aroma. Table 5 shows the sensory similarity between the target aroma and each evaluation sample.

TABLE 5
Sensory similarity between target
aroma and each evaluation sample

| Test | Sample | Similarity | Corresponding evaluation labels |
|---|---|---|---|
| 1 | Target | 4.5 | “relatively similar” |
| | | | or “very similar” |
| | Reconstructed | 4.0 | “relatively similar” |
| | product | | |
| 2 | Target | 5.0 | “very similar” |
| | Reconstructed | 3.5 | “neither similar |
| | product | | nor dissimilar” or |
| | | | “relatively similar” |

By reconstruction of the target aroma using the olfactory receptor activity profiles as an index, a reconstruction accuracy of about 70 to 90% was achieved for the targets presented in a blind manner.

### <3-2> Aroma Reconstruction Targeting a Single Aroma Component

The four types of substances shown in Table 6 were selected and used as target aromas. Olfactory receptor activity profiles of the targets in an olfactory receptor activity database were set as target profiles.

TABLE 6

| Target (single component) | CAS number |
|---|---|
| (−)-Carvone | 6485-40-1 |
| trans-Anethole | 4180-23-8 |
| Syringol | 91-10-1 |
| Sotolon | 28664-35-9 |

For each target, 196 types of substances obtained by excluding the target from the olfactory receptor activity database were used as a test substance group, and olfactory receptor activity profiles of these test substances were set as a test substance group profile.

Using the target profile as an objective variable (352 points×1 variable), and the test substance group profile as explanatory variables (352 points×196 variables), nonnegative constraint linear sparse modeling was performed to obtain a composite profile that approximates the target profile, and coefficients of the variables (the combination ratios of the receptor activity profiles of two or more target substances that provide the composite profile). Specifically, a LASSO (least absolute shrinkage and selection operator) regression model was used. The intercept of the model was set to 0. Using a parameter λ that defines the strength of regularization, optimization was performed such that the number of variables having a coefficient greater than 0 (the number of two or more target substances that provide the composite profile) was not more than 4. A mixing ratio of the target substances was determined according to the ratio of the coefficients of the model.

The target substances were mixed according to the calculated mixing ratio, to prepare a reconstructed product. For sensory evaluation, each of the target and the reconstructed product was diluted to a concentration at which the aroma could be smelled without difficulty. Table 7 shows the compositions of the evaluation samples used for the sensory evaluation.

TABLE 7
Compositions of evaluation samples used for sensory evaluation

| | | Test | CAS | Evaluation | |
|---|---|---|---|---|---|
| Test | Sample | substance | number | concentration | |
| 1 | Target | (−)-carvone | 6485-40-1 | 100.0 | ppm |
| | Reconstructed | trans-anethole | 4180-23-8 | 54.7 | ppm |
| | product | carveol | 99-48-9 | 28.8 | ppm |
| | | gamma- | 104-50-7 | 10.7 | ppm |
| | | octalactone | | | |
| | | 2-pentylfuran | 3777-69-3 | 5.8 | ppm |
| 2 | Target | trans-anethole | 4180-23-8 | 100.0 | ppm |
| | Reconstructed | (−)-carvone | 6485-40-1 | 53.9 | ppm |
| | product | hexanol | 111-27-3 | 18.4 | ppm |
| | | gamma- | 104-50-7 | 15.9 | ppm |
| | | octalactone | | | |
| | | benzaldehyde | 100-52-7 | 11.8 | ppm |
| 3 | Target | syringol | 91-10-1 | 100.0 | ppm |
| | Reconstructed | eugenol | 97-53-0 | 38.7 | ppm |
| | product | guaiacol | 90-05-1 | 32.3 | ppm |
| | | 4-ethylguaiacol | 2785-89-9 | 29.0 | ppm |
| 4 | Target | sotolon | 28664-35-9 | 100.0 | ppm |
| | Reconstructed | methyl | 80-71-7 | 39.8 | ppm |
| | product | cyclopentenolone | | | |
| | | 2-hydroxy- | 698-10-2 | 34.5 | ppm |
| | | 3-methyl- | | | |

| 2-hexen-4-olide | | | |
|---|---|---|---|
| delta- | 2721-22-4 | 20.2 | ppm |
| tetradecalactone | | | |
| sclareol | 515-03-7 | 5.5 | ppm |

The sensory evaluation was carried out in a blind manner (n=4×3 times). A target aroma was presented to each panelist as a reference sample. Subsequently, as an evaluation sample, the target aroma or the aroma of the reconstructed product was presented in a blind manner. Each panelist evaluated how similar the quality of aroma of each evaluation sample was to the quality of aroma of the reference sample, using a 5-point scale (0 point: not similar at all, 1 point: slightly similar, 2 points: moderately similar, 3 points: very similar, 4 points: extremely similar). Each test was carried out by each subject in three replicates on different days. For each evaluation sample, the median of the scores in all replicates by all panelists was calculated to determine the “sensory similarity” to the target aroma. Table 8 shows the sensory similarity between the target aroma and each evaluation sample.

TABLE 8
Sensory similarity between target
aroma and each evaluation sample

| Test | Sample | Similarity | Corresponding evaluation labels |
|---|---|---|---|
| 1 | Target | 4.0 | “extremely similar” |
| | Reconstructed | 3.0 | “moderately similar” |
| | product | | or “very similar” |
| 2 | Target | 4.0 | “extremely similar” |
| | Reconstructed | 3.0 | “moderately similar” |
| | product | | or “very similar” |
| 3 | Target | 4.0 | “extremely similar” |
| | Reconstructed | 4.0 | “very similar” |
| | product | | or “extremely similar” |
| 4 | Target | 4.0 | “extremely similar” |
| | Reconstructed | 4.0 | “extremely similar” |
| | product | | |

By the reconstruction of the target aroma using the olfactory receptor activity profiles as an index, similarly to the case using the group of 3103 types of test substances, the case using the group of 196 types of test substances resulted in achievement of a reconstruction accuracy of about 70 to 100% for the targets presented in a blind manner.

### <3-3> Aroma Reconstruction Targeting a Composite Aroma

The three types of aroma oils shown in Table 9 were selected as target aromas. Olfactory receptor activity profiles of the targets in an olfactory receptor activity database were set as target profiles.

TABLE 9

| Target (mixture) | Distributor | Product number |
|---|---|---|
| Peppermint essential oil | Tree of Life | 08-440-3720 |
| Frankincense olibanum/mastic essential | Tree of Life | 08-440-3170 |
| oil | | |
| Natural essential oil “Gekka Bijin” | Herbleap | 0335 |

For each target, 332 types of substances obtained by excluding the target from the olfactory receptor activity database were used as a test substance group, and olfactory receptor activity profiles of these test substances were set as a test substance group profile.

Using the target profile as an objective variable (352 points×1 variable), and the test substance group profile as explanatory variables (352 points×332 variables), nonnegative constraint linear sparse modeling was performed to obtain a composite profile that approximates the target profile, and coefficients of the variables (the combination ratios of the receptor activity profiles of two or more target substances that provide the composite profile). Specifically, a LASSO (least absolute shrinkage and selection operator) regression model was used. The intercept of the model was set to 0. Using a parameter λ that defines the strength of regularization, optimization was performed such that the number of variables having a coefficient greater than 0 (the number of two or more target substances that provide the composite profile) was not more than 4. A mixing ratio of the target substances was determined according to the ratio of the coefficients of the model.

The target substances were mixed according to the calculated mixing ratio, to prepare a reconstructed product. For sensory evaluation, each of the target and the reconstructed product was diluted to a concentration at which the aroma could be smelled without difficulty. Table 10 shows the compositions of the evaluation samples used for the sensory evaluation.

TABLE 10
Compositions of evaluation samples used for sensory evaluation

| | | | | Evaluation | |
|---|---|---|---|---|---|
| Test | Sample | Test substance | CAS number | concentration | |
| 1 | Target | Peppermint | | 100% | |
| | | essential oil | | | |
| | Reconstructed | 2-Pentylfuran | 3777-69-3 | 37.1 | ppm |
| | product | (+)-Carvone | 2244-16-8 | 34.0 | ppm |
| | | (−)-Carvone | 6485-40-1 | 17.0 | ppm |
| | | Carvacrol | 499-75-2 | 11.9 | ppm |
| 2 | Target | Frankincense | | 100% | |
| | | olibanum/mastic | | | |
| | | essential oil | | | |
| | Reconstructed | (+)-Carvone | 2244-16-8 | 35.9 | ppm |
| | product | (−)-Carvone | 6485-40-1 | 33.6 | ppm |
| | | 2-Pentylfuran | 3777-69-3 | 22.9 | ppm |
| | | 4-Isopropylbenzyl | 536-60-7 | 7.7 | ppm |
| | | Alcohol | | | |
| 3 | Target | Natural | | 100% | |
| | | essential oil | | | |
| | | “Gekka Bijin” | | | |
| | Reconstructed | gamma- | 104-61-0 | 49.3 | ppm |
| | product | Nonalactone | | | |
| | | Eugenol | 97-53-0 | 36.8 | ppm |
| | | 6-Methyl-5- | 110-93-0 | 12.0 | ppm |
| | | hepten-2-one | | | |
| | | (+)-Carvone | 2244-16-8 | 1.9 | ppm |

The sensory evaluation was carried out with n=4. A target aroma was presented to each panelist as a reference sample. Subsequently, the aroma of the reconstructed product was presented as an evaluation sample. Each panelist evaluated how similar the quality of aroma of each evaluation sample was to the quality of aroma of the reference sample, using a 5-point scale (0 point: not similar at all, 1 point: slightly similar, 2 points: moderately similar, 3 points: very similar, 4 points: extremely similar). For each evaluation sample, the median of the scores given by all panelists was calculated and used as a “sensory similarity” to the target aroma. Table 11 shows the sensory similarity between the target aroma and each evaluation sample.

TABLE 11
Sensory similarity between target
aroma and each evaluation sample

| Test | Sample | Similarity | Corresponding evaluation labels |
|---|---|---|---|
| 1 | Reconstructed | 2.5 | “moderately similar” |
| | product | | or “very similar” |
| 2 | Reconstructed | 2.0 | “moderately similar” |
| | product | | |
| 3 | Reconstructed | 2.0 | “moderately similar” |
| | product | | |

Even in the cases where the aroma of a mixture was used as the target, reconstruction of the aroma using the olfactory receptor activity profiles as an index resulted in achievement of a reconstruction accuracy ranging from moderate similarity to very high similarity.

### <4> Reconstruction of a Target Aroma Using a Reporter Signal as an Index

Reconstruction of the target aroma and sensory evaluation were carried out in the same manner as in <3-1> except that, as an index, reporter signals corrected with an internal standard described in the manufacturer's protocol were used instead of the corrected olfactory receptor activity profiles.

The two types of substances shown in Table 3 were selected and used as target aromas. The reporter signals of the targets in an olfactory receptor activity database were set as target profiles.

For each target, 3102 types of substances obtained by excluding the target from the olfactory receptor activity database were used as a test substance group, and reporter signals of these test substances were set as a test substance group profile.

Using the target profile as an objective variable (352 points×1 variable), and the test substance group profile as explanatory variables (352 points×3102 variables), nonnegative constraint linear sparse modeling was performed to obtain a composite profile that approximates the target profile, and coefficients of the variables (combination ratios of reporter signals of two or more target substances that provide the composite profile). Specifically, a LASSO (least absolute shrinkage and selection operator) regression model was used. The intercept of the model was set to 0. Using a parameter λ that defines the strength of regularization, optimization was performed such that the number of variables having a coefficient greater than 0 (the number of two or more target substances that provide the composite profile) was not more than 4. A mixing ratio of the target substances was determined according to the ratio of the coefficients of the model.

The target substances were mixed according to the calculated mixing ratio to prepare reconstructed product 2. As a control group, reconstructed product 1 was prepared by calculation using the olfactory receptor activity profiles as an index by the method described in <3-1>. For sensory evaluation, each of the target, reconstructed product 1, and reconstructed product 2 was diluted to a concentration at which the aroma could be smelled without difficulty. Table 12 shows the compositions of the evaluation samples used for the sensory evaluation.

TABLE 12
Compositions of evaluation samples used for sensory evaluation

| | | | | Evaluation |
|---|---|---|---|---|
| | | Test | CAS | concen- |
| Test | Sample | substance | number | tration |
| 1 | Target | Syringol | 91-10-1 | 0.140% |
| | Reconstructed | Guaiacol | 90-05-1 | 0.049% |
| | product 1 | 4-Ethylguaiacol | 2785-89-9 | 0.037% |
| | | 4-Vinylguaiacol | 7786-61-0 | 0.027% |
| | | 2-methoxy-5- | 1195-09-1 | 0.013% |
| | | methylphenol | | |
| | Reconstructed | Propyl | 121-79-9 | 0.155% |
| | product 2 | gallate | | |
| | | 2- | 120-75-2 | 0.023% |
| | | methylbenzothiazole | | |
| | | 2,5-Xylenol | 95-87-4 | 0.002% |
| | | Nerolin Yara Yara | 93-04-9 | 0.001% |
| 2 | Target | trans,trans-2,4- | 25152-84-5 | 0.138% |
| | | Decadienal | | |
| | Reconstructed | trans,trans-2,4- | 5910-87-2 | 0.111% |
| | product 1 | Nonadienal | | |
| | | 3- | 620-22-4 | 0.013% |
| | | methylbenzonitrile | | |
| | Reconstructed | Propyl | 121-79-9 | 0.109% |
| | product 2 | gallate | | |
| | | trans-2-Nonenal | 18829-56-6 | 0.049% |
| | | Acetic acid | 64-19-7 | 0.002% |
| | | (4Z,7Z)-decadienol | 104188-11-6 | 0.004% |

The sensory evaluation was carried out in a blind manner (n=8). A target aroma was presented to each panelist as a reference sample. Subsequently, as an evaluation sample, the target aroma, the aroma of reconstructed product 1, or the aroma of reconstructed product 2 was presented in a blind manner in a random order. Each panelist evaluated how similar the quality of aroma of each evaluation sample was to the quality of aroma of the reference sample, using a 5-point scale (1 point: not similar at all, 2 points: rather dissimilar, 3 points: neither similar nor dissimilar, 4 points: relatively similar, 5 points: very similar). For each evaluation sample, the median of the scores given by all panelists was calculated and used as a “sensory similarity” to the target aroma. Table 13 shows the sensory similarity between the target aroma and each evaluation sample.

TABLE 13
Sensory similarity between target
aroma and each evaluation sample

| Test | Sample | Similarity | Corresponding evaluation labels |
|---|---|---|---|
| 1 | Target | 4.5 | “relatively similar” |
| | | | or “very similar” |
| | Reconstructed | 3.0 | “neither similar |
| | product 1 | | nor dissimilar” |
| | Reconstructed | 1.0 | “not similar |
| | product 2 | | at all” |
| 2 | Target | 4.0 | “relatively similar” |
| | Reconstructed | 3.0 | “neither similar |
| | product 1 | | nor dissimilar” |
| | Reconstructed | 1.5 | “not similar at all” or |
| | product 2 | | “rather dissimilar” |

In the sensory evaluation, the formulations based on the calculation using the olfactory receptor activity profiles as an index showed higher degrees of similarity than the formulations based on the calculation using raw measurement values of the reporter signals as an index.

### <5> Calculation of Minimum Set for Reproducing Diverse Aromas

In order to reproduce diverse aromas with a limited number of fragrance cartridges, a minimum set was calculated using the present invention. Specifically, for reproducing a target aroma set of 72 items shown in Table 14 by mixing 12 fragrance cartridges, the composition of each cartridge and the mixing ratio thereof were calculated.

TABLE 14
Target Aroma Set (72 Items)

| | |
|---|---|
| 01 | Grape gummy KJ-G |
| 02 | Satsuma mandar in gummy KJ-O |
| 03 | Muscat gummy KJ-M |
| 04 | Grape gummy PU-G |
| 05 | Muscat gummy PU-M |
| 06 | Lemon gummy PU-L |
| 07 | Plum gummy PU-U |
| 08 | Cola gunmy TG-C |
| 09 | Energy drink gummy TG-E |
| 10 | Soda gummy TG-S |
| 11 | Strawberry gummy CL-S |
| 12 | Orange gummy CL-O |
| 13 | Lemon gummy CL-L |
| 14 | Muscat gummy CL-M |
| 15 | Green apple gummy CL-A |
| 16 | Blueberry gummy CL-B |
| 17 | Grape gummy CL-G |
| 18 | Premium strawberry gummy PU-S |
| 19 | Green apple gummy KJ-A |
| 20 | Peach gummy KJ-P |
| 21 | Strawberry gummy KJ-S |
| 22 | Nijisseiki pear gummy ZN-N |
| 23 | Shine Muscat gummy ZN-M |
| 24 | Lemongrass aroma oil |
| 25 | Rose aroma oil |
| 26 | Grapefruit aroma oil |
| 27 | Rosemary aroma oil |
| 28 | Vanilla aroma oil |
| 29 | Jasmine aroma oil |
| 30 | Coffee aroma oil |
| 31 | Daphne aroma oil |
| 32 | Lemon aroma oil |
| 33 | Sandalwood aroma oil |
| 34 | Peach aroma oil |
| 35 | Perilla aroma oil |
| 36 | Osmanthus aroma oil |
| 37 | Mint aroma oil |
| 38 | Heliotrope aroma oil |
| 39 | Lavender aroma oil |
| 40 | Strawberry aroma oil |
| 41 | Frankincense aroma oil |
| 42 | Cacao aroma oll |
| 43 | Lily aroma oil |
| 44 | Ginger aroma oil |
| 45 | Cherry blossom aroma oil |
| 46 | Linden blossom aroma oil |
| 47 | Japanese cypress aroma oil |
| 48 | Epiphyllum oxypetalum aroma oil |
| 49 | Vanilla essence |
| 50 | Lemon essence |
| 51 | Milk essence |
| 52 | Yogurt essence |
| 53 | Coffee essence |
| 54 | Chocolate essence |
| 55 | Cocoa essence |
| 56 | Oolong tea essence |
| 57 | Honey essence |
| 58 | Custard cream flavor |
| 59 | Roasted green tea flavor |
| 60 | Lean beef flavor |
| 61 | Fatty beef flavor |
| 62 | Lean/fatty beef flavor |
| 63 | (−)-carvone |
| 64 | anethole |
| 65 | diallyl disulfide |
| 66 | 2-propylpyrazine |
| 67 | 2,6-dimethoxyphenol |
| 68 | (E,E)-2,4-decadienal |
| 69 | 2-ethyl-3-methylpyrazine |
| 70 | (2E)-3,7-dimethyl-2,6-octadienal |
| 71 | 3-hydroxy-4,5-dimethyl-2(5H)-furanone |
| 72 | butanoic acid |

### <5-1> Reconstruction of Target Aroma Set

In the same manner as described in <3-3> above, a mixing ratio for reconstructing each target profile of the target aroma set with not more than 40 aroma components was calculated. As a result, a matrix (72 rows×315 columns) representing the mixing ratios of aroma components for reconstructing the 72 items of the target aroma set was obtained. This matrix is hereinafter referred to as the reconstruction recipe matrix W. The average value of correlation coefficients between the target profiles and the composite profiles of the reconstructed products for the 72 items of the target aroma set was r=0.647 (standard deviation, 0.091; minimum value, 0.421; maximum value, 0.807).

### <5-2> Approximate Decomposition of the Reconstruction Recipe Matrix

For the reconstruction recipe matrix W, nonnegative matrix factorization (NMF) was performed with a dimensionality of 12 to decompose the matrix into the form W≅AH. As a result, a basis matrix A (72 rows×12 columns) and a coefficient matrix H (12 rows×315 columns) were obtained. In this case, the coefficient matrix H represents the mixing ratios of the aroma components for constituting each cartridge of the minimum set, and the basis matrix A represents the mixing ratios of the cartridges for reconstructing the target aroma set. The correlation coefficient between the reconstruction recipe matrix W and its approximation matrix AH was r=0.901, which indicates good approximation accuracy.

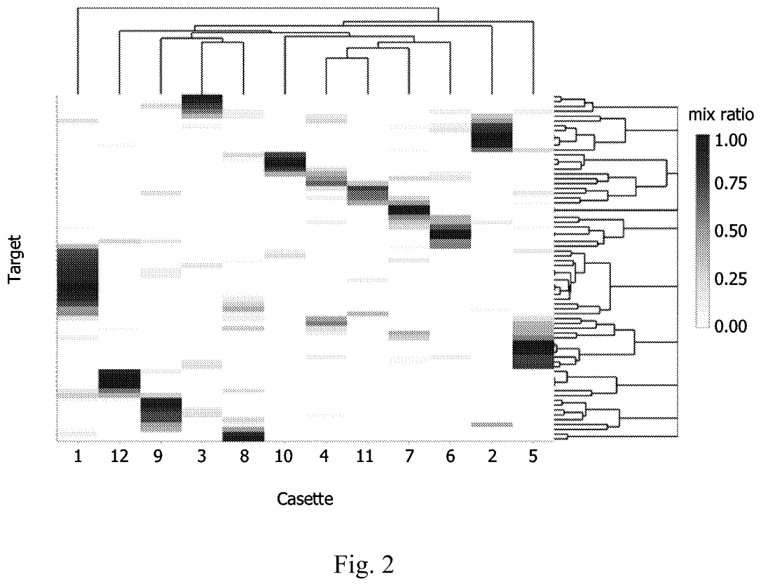

FIG. 2 shows the basis matrix A. In FIG. 2, the 72 items of the target aroma set are arranged vertically, and the 12 fragrance cartridges are arranged horizontally. The mixing amounts of the fragrance cartridges used for reconstruction of each target aroma are represented as a heat map. A darker color represents a larger mixing amount. The rows and columns are arranged according to results of hierarchical clustering analysis, respectively.

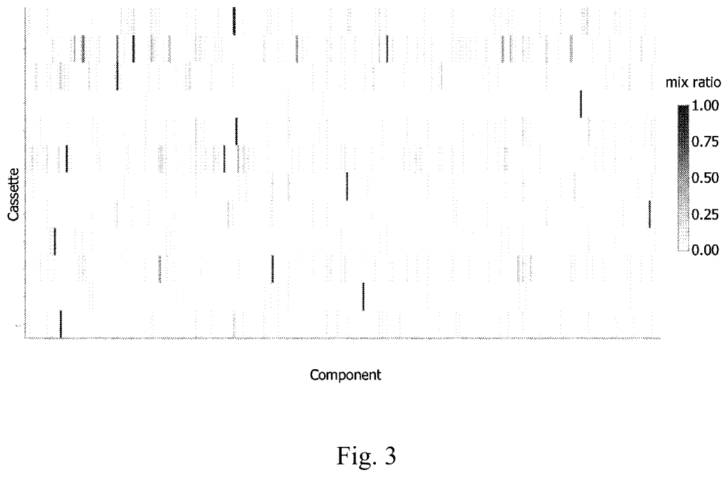

FIG. 3 shows the coefficient matrix H. In FIG. 3, the 12 fragrance cartridges are arranged vertically, and the 315 aroma components are arranged horizontally. The mixing amounts of the aroma components used for reconstruction of each fragrance cartridge are represented as a heat map. A darker color represents a larger mixing amount.

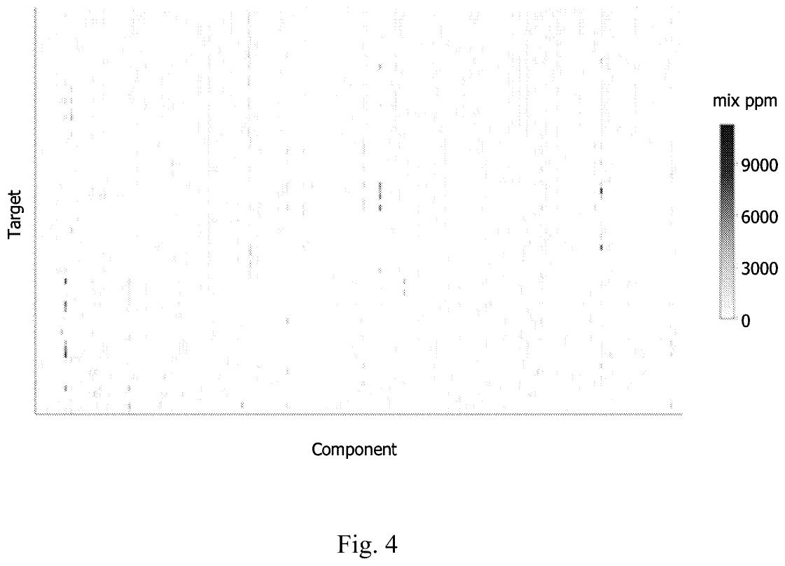

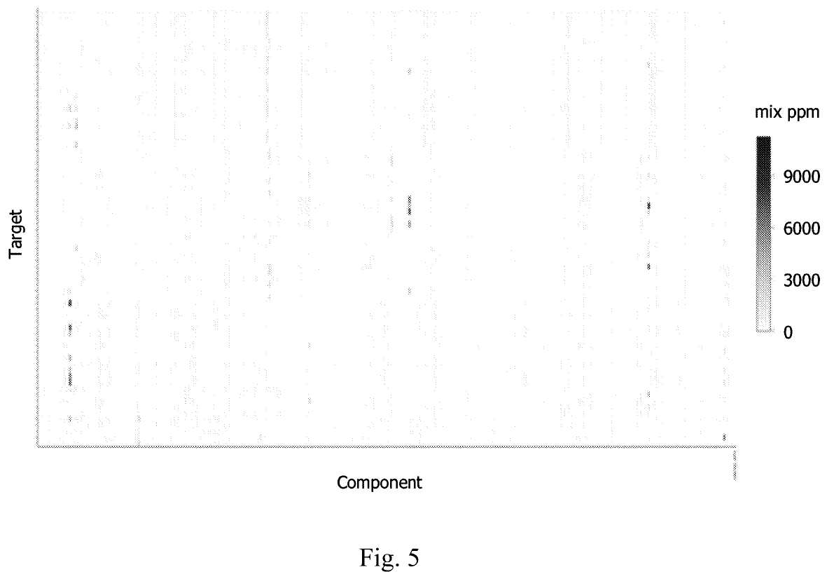

FIG. 4 shows the reconstruction recipe matrix W. FIG. 5 shows the approximation matrix AH. In FIG. 4 and FIG. 5, the 72 items of the target aroma set are arranged vertically, and the 315 aroma components are arranged horizontally. The mixing amounts of the aroma components used for reconstruction of each target aroma are represented as a heat map. A darker color represents a larger mixing amount.

Table 15 shows, for each of the 12 fragrance cartridges, the number of aroma components constituting the cartridge.

TABLE 15
Number of aroma components used for
constituting each fragrance cartridge

| Fragrance | Number of |
|---|---|
| cartridge | components |
| 01 | 137 |
| 02 | 134 |
| 03 | 100 |
| 04 | 64 |
| 05 | 118 |
| 06 | 128 |
| 07 | 78 |
| 08 | 75 |
| 09 | 97 |
| 10 | 115 |
| 11 | 65 |
| 12 | 104 |

### <6> Optimization of the Number of Fragrance Cartridges

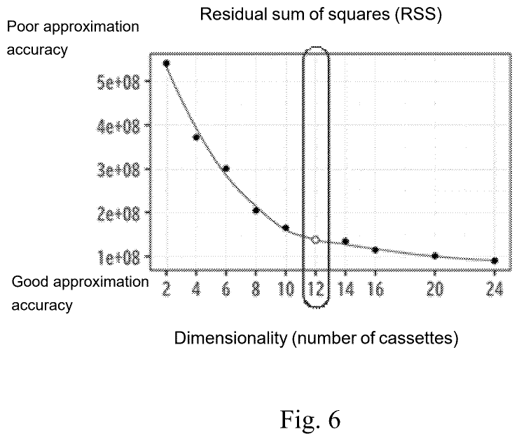

FIG. 6 shows the relationship between the number of fragrance cartridges of the minimum set and the approximation accuracy (residual sum of squares, RSS) by the approximation matrix AH, wherein the number of fragrance cartridges was set to 2, 4, 6, 8, 10, 12, 14, 16, 20, or 24

FIG. 6 is a graph representing the relationship between the number of fragrance cartridges of the minimum set and the approximation accuracy by the approximation matrix AH. In FIG. 6, the number of fragrance cartridges (dimensionality of NMF) was plotted on the horizontal axis, and the residual sum of squares (RSS), which serves as an index of the approximation accuracy between the reconstruction recipe matrix W and the approximation matrix AH, was plotted on the vertical axis. The open circle indicates the inflection point of the decrease in RSS.

As shown in FIG. 6, an increase in the number of fragrance cartridges resulted in a decrease in RSS and improvement in the approximation accuracy. The number of fragrance cartridges was 12 at the inflection point, where the slope of the decrease in RSS due to the increase in the number of fragrance cartridges became gentle. This means that the approximation accuracy is hardly improved even by increasing the number of fragrance cartridges to 12 or more. In other words, taking into account the cost required for increasing output ports of an odor presentation device or adding fragrance cartridges, the optimum number of fragrance cartridges can be said to be 12 for the reconstruction of the target aroma set shown in the present Example.

### <7> Calculation of Minimum Set for Reproducing Diverse Aromas (Alternative Method)

In <5> above, first, the mixing ratios of the aroma components for reconstructing the 72 items of the target aroma set (reconstruction recipe matrix) were calculated <5-1>, and then the compositions of the fragrance cartridges for reconstructing the target aroma set were obtained by nonnegative matrix factorization (NMF) <5-2>. Theoretically, this procedure can be reversed. Specifically, first, the target profile matrix of the 72 items of the target aroma set is subjected to nonnegative matrix factorization (NMF) to obtain a profile (fragrance profile set) matrix of the fragrance cartridges. Subsequently, each profile of the fragrance profile set is used as a target profile, and the mixing ratio of the aroma components for reconstructing the profile is calculated in the method.

### <7-1> Approximate Decomposition of a Target Profile Matrix of a Target Aroma Set

Since the activity values obtained in <2-2> above contained negative values and could not be subjected to nonnegative matrix factorization (NMF), the activity values were converted into nonnegative values by an exponential function. Subsequent analyses were carried out using the resulting nonnegative activity profile. Depending on the measurement method and the standardization, the activity values may be nonnegative. In this case, the nonnegative conversion is not necessary. Moreover, as long as the values can be converted to nonnegative values, the conversion is not limited to an exponential function. For the target profile matrix W (72 rows×355 columns) of the target aroma set, nonnegative matrix factorization (NMF) was performed with a dimensionality of 12 to decompose the matrix into the form W≅AH. As a result, a basis matrix A (72 rows×12 columns) and a coefficient matrix H (12 rows×355 columns) were obtained.

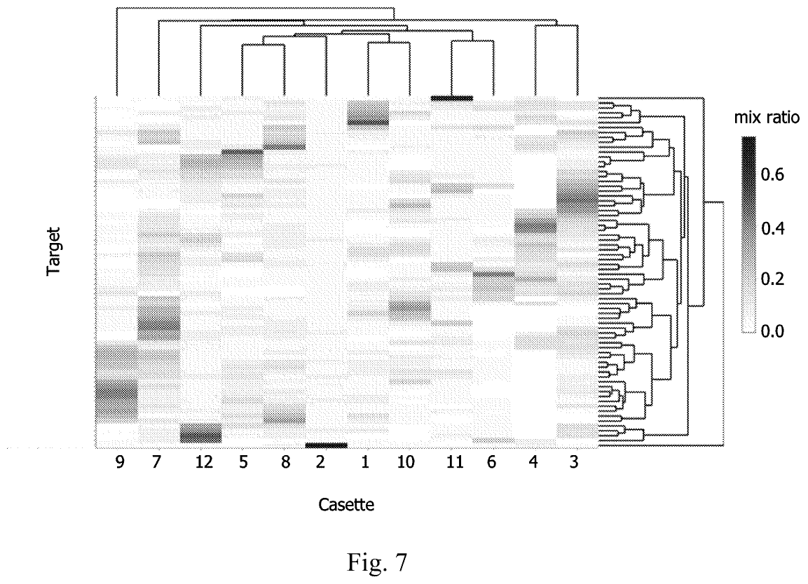

FIG. 7 shows the basis matrix A. In FIG. 7, the 72 items of the target aroma set are arranged vertically, and the 12 fragrance cartridges are arranged horizontally. The mixing amounts of the fragrance cartridges used for reconstruction of each target aroma are represented as a heat map. A darker color represents a larger mixing amount. The rows and columns are arranged according to the results of hierarchical clustering analysis, respectively.

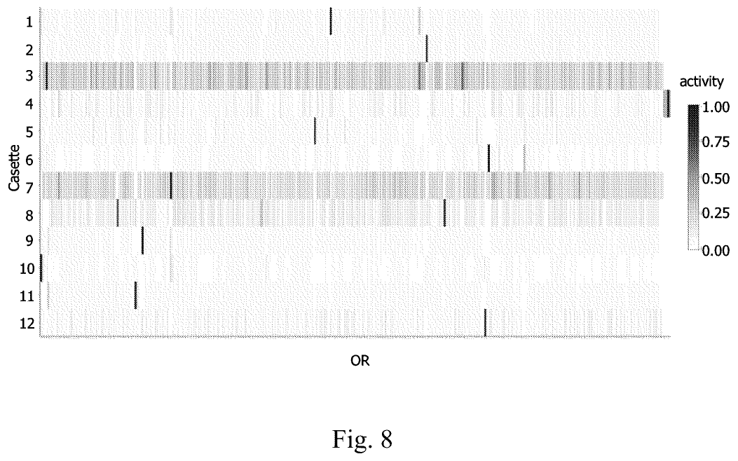

FIG. 8 shows the coefficient matrix H. In FIG. 8, the 12 fragrance cartridges are arranged vertically, and the 355 olfactory receptors are arranged horizontally. The receptor (OR) activity profile of each fragrance cartridge is represented as a heat map. A darker color represents stronger activity.

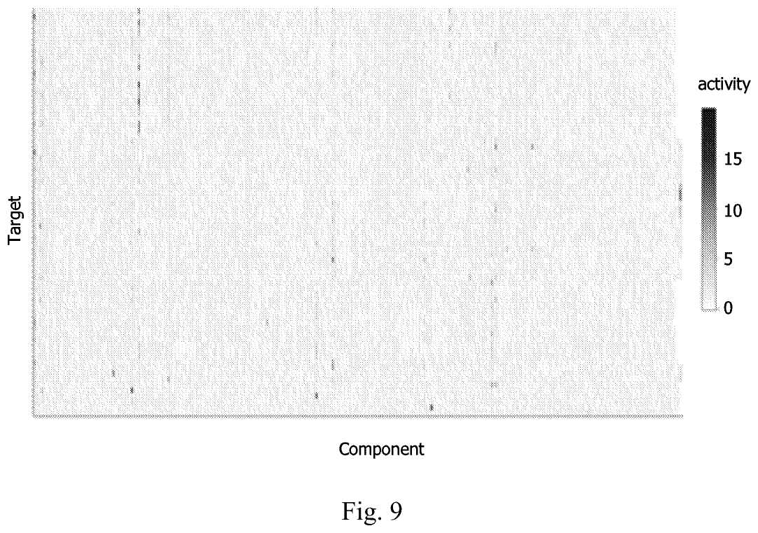

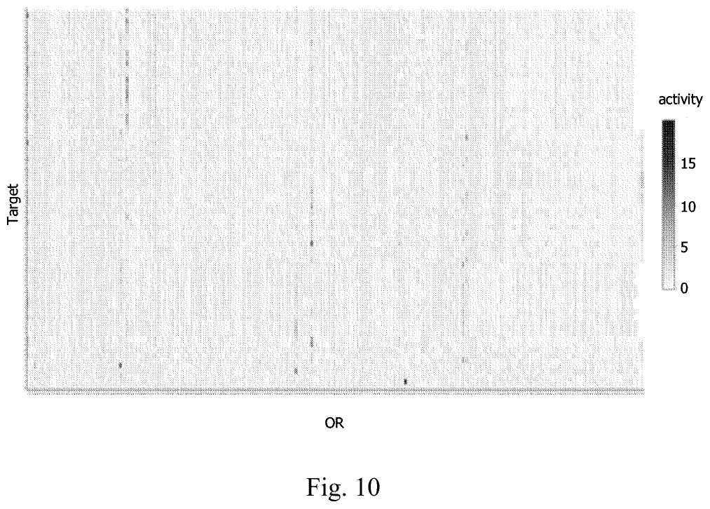

FIG. 9 shows the target profile matrix W. FIG. 10 shows the approximation matrix AH. In FIG. 9 and FIG. 10, the 72 items of the target aroma set are arranged vertically, and the 355 olfactory receptors are arranged horizontally. The target profile and the approximate profile of each target aroma are represented as a heat map. A darker color represents stronger activity.

In this case, the coefficient matrix H corresponds to the fragrance profile set, and the basis matrix A corresponds to the mixing ratios of the fragrance cartridges for reconstructing the target aroma set. The correlation coefficient between the target profile matrix W and its approximation matrix AH was r=0.883, which indicates good approximation accuracy.

### <7-2> Reconstruction of Fragrance Cartridges

For the fragrance profile set (coefficient matrix H) obtained in <7-1> above, a mixing ratio for reconstructing each profile with not more than 40 aroma components was calculated. As a result, a matrix (12 rows×44 columns) representing the mixing ratios of the aroma components for reconstructing the 12 fragrance cartridges was obtained. The average value of correlation coefficients between the target profiles and the composite profiles of the reconstructed products for the 12 fragrance cartridges was r=0.824 (standard deviation, 0.147; minimum value, 0.578; maximum value, 0.994).

Table 16 shows, for each of the 12 fragrance cartridges, the number of aroma components constituting the cartridge.

TABLE 16
Number of aroma components used for
constituting each fragrance cartridge

| Fragrance | Number of |
|---|---|
| cartridge | components |
| 01 | 3 |
| 02 | 1 |
| 03 | 16 |
| 04 | 5 |
| 05 | 1 |
| 06 | 1 |
| 07 | 8 |
| 08 | 6 |
| 09 | 2 |
| 10 | 1 |
| 11 | 1 |
| 12 | 2 |

1. A method of producing a formulation that exhibits a target sensory quality, the method comprising:
identifying two or more target substances and a combination ratio of receptor activity profiles of the target substances such that the target substances and the combination ratio provide a composite profile that approximates a target profile,
wherein each of the receptor activity profiles is a receptor activity profile for two or more receptors,
wherein the target profile is a receptor activity profile corresponding to the target sensory quality for the receptors, and
wherein a formulation of the target substances with the combination ratio is regarded as the formulation that exhibits the target sensory quality; and
producing a formulation of the target substances that exhibits the target sensory quality by blending the target substances according to the combination ratio.

2. The method according to claim 1, wherein the target substances comprise 2 to 8 types of substances.

3. The method according to claim 1, wherein, in the step, the target substances are selected from a test substance group.

4. The method according to claim 3, wherein the test substance group comprises more types of substances than the target substances.

5. The method according to claim 3, wherein the test substance group comprises not less than 100 types of substances.

6. The method according to claim 3, wherein, in the step, the target profile is used as an objective variable, and the receptor activity profile, for the receptors, of each substance constituting the test substance group is used as an explanatory variable, and wherein the step is carried out by nonnegative constraint sparse modeling.

7. The method according to claim 6, wherein the sparse modeling is carried out using a LASSO regression model.

8. The method according to claim 1, wherein the step is carried out such that an error between the target profile and the composite profile is not more than 0.7 in terms of a weighted RMSE.

9. The method according to claim 1, wherein the receptors comprise not less than 300 types of receptors.

10. The method according to claim 1, wherein the receptors are selected from the group consisting of G protein-coupled receptors and ion channel-type receptors.

11. The method according to claim 1, wherein the receptors are selected from the group consisting of olfactory receptors and taste receptors.

12. The method according to claim 1, wherein not less than 50% of the total number of receptors are selected from the group consisting of OR1A1, OR1A2, OR1B1, OR1C1, OR1D2, OR1D5, OR1E1, OR1F1, OR1F12, OR1G1, OR1I1, OR1J1, OR1J2, OR1J4, OR1K1, OR1L1, OR1L3, OR1L4, OR1L8, OR1M1, OR1N1, OR1N2, OR1Q1, OR1R1P, OR1S1, OR2A1, OR2A2, OR2A4, OR2A5, OR2A12, OR2A14, OR2A25, OR2AE1, OR2AG1, OR2AG2, OR2AJ1P, OR2AK2, OR2AP1, OR2AT4, OR2B2, OR2B3, OR2B6, OR2B11, OR2C1, OR2C3, OR2D2, OR2D3, OR2F1, OR2G2, OR2G3, OR2G6, OR2H1, OR2H2, OR2J2, OR2J3, OR2K2, OR2L2, OR2L8, OR2L13, OR2M2, OR2M4, OR2M7, OR2S2, OR2T1, OR2T2, OR2T5, OR2T6, OR2T8, OR2T10, OR2T11, OR2T27, OR2T34, OR2V2, OR2W1, OR2W3, OR2Y1, OR2Z1, OR3A1, OR3A2, OR3A3, OR3A4, OR4A5, OR4A15, OR4A16, OR4A47, OR4B1, OR4C3, OR4C5, OR4C6, OR4C11, OR4C12, OR4C13, OR4C15, OR4C16, OR4C46, OR4D1, OR4D2, OR4D5, OR4D6, OR4D9, OR4D10, OR4D11, OR4E2, OR4F3, OR4F5, OR4F6, OR4F14P, OR4F15, OR4G11P, OR4H12P, OR4K1, OR4K2, OR4K5, OR4K13, OR4K14, OR4K15, OR4K17, OR4L1, OR4M1, OR4N2, OR4N4, OR4N5, OR4P4, OR4Q3, OR4S1, OR4S2, OR4X1, OR4X2, OR5A1, OR5A2, OR5AC2, OR5AK2, OR5AK3P, OR5AN1, OR5AP2, OR5AR1, OR5AS1, OR5AU1, OR5B2, OR5B3, OR5B12, OR5B17, OR5B21, OR5C1, OR5D13, OR5D14, OR5D16, OR5D18, OR5F1, OR5H1, OR5H2, OR5H6, OR5H14, OR5I1, OR5J2, OR5K1, OR5K3, OR5K4, OR5L2, OR5M3, OR5M8, OR5M9, OR5M10, OR5M11, OR5P3, OR5R1, OR5T1, OR5T2, OR5T3, OR5V1, OR5W2, OR6A2, OR6B1, OR6B2, OR6C1, OR6C2, OR6C3, OR6C4, OR6C6, OR6C65, OR6C66P, OR6C68, OR6C70, OR6C74, OR6C75, OR6C76, OR6F1, OR6J1, OR6K2, OR6K3, OR6K6, OR6M1, OR6N1, OR6N2, OR6P1, OR6Q1, OR6S1, OR6T1, OR6V1, OR6X1, OR6Y1, OR7A3P, OR7A5, OR7A10, OR7A17, OR7C1, OR7C2, OR7D2, OR7D4, OR7E24, OR7G1, OR7G2, OR7G3, OR8A1, OR8B3, OR8B4, OR8B8, OR8B12, OR8D1, OR8D2, OR8D4, OR8G2, OR8G5, OR8H3, OR8I2, OR8J1, OR8J3, OR8K1, OR8K3, OR8K5, OR8S1, OR8U1, OR9A4, OR9G1, OR9G4, OR9I1, OR9K2, OR9Q1, OR9Q2, OR10A3, OR10A4, OR10A5, OR10A6, OR10A7, OR10AD1, OR10AG1, OR10C1, OR10D3, OR10D4P, OR10G2, OR10G3, OR10G4, OR10G6, OR10G7, OR10G9, OR10H2, OR10H4, OR10J1, OR10J3, OR10J5, OR10K1, OR10K2, OR10P1, OR10Q1, OR10R2, OR10S1, OR10T2, OR10V1, OR10W1, OR10X1, OR10Z1, OR11A1, OR11G2, OR11H4, OR11H6, OR11H12, OR11L1, OR12D2, OR12D3, OR13A1, OR13C2, OR13C3, OR13C4, OR13C8, OR13D1, OR13F1, OR13G1, OR13H1, OR13J1, OR14A2, OR14A16, OR14C36, OR14I1, OR14J1, OR14K1, OR14L1P, OR51A1P, OR51A4, OR51A7, OR51B2, OR51B4, OR51B5, OR51B6, OR51D1, OR51E1, OR51E2, OR51F1, OR51F2, OR51F5P, OR51G1, OR51G2, OR51H1, OR51I1, OR51I2, OR51L1, OR51M1, OR51Q1, OR51S1, OR51T1, OR51V1, OR52A1, OR52A4, OR52A5, OR52B2, OR52B4, OR52B6, OR52D1, OR52E2, OR52E4, OR52E5, OR52E8, OR52H1, OR52I2, OR52J3, OR52K2, OR52L2P, OR52M1, OR52N1, OR52N2, OR52N4, OR52N5, OR52P2P, OR52R1, OR52W1, OR52Z1P, OR56A1, OR56A3, OR56A4, OR56A5, OR56B1, OR56B2P, and OR56B4.

13. The method according to claim 1, wherein the receptors comprise not less than 50% of OR1A1, OR1A2, OR1B1, OR1C1, OR1D2, OR1D5, OR1E1, OR1F1, OR1F12, OR1G1, OR1I1, OR1J1, OR1J2, OR1J4, OR1K1, OR1L1, OR1L3, OR1L4, OR1L8, OR1M1, OR1N1, OR1N2, OR1Q1, OR1R1P, OR1S1, OR2A1, OR2A2, OR2A4, OR2A5, OR2A12, OR2A14, OR2A25, OR2AE1, OR2AG1, OR2AG2, OR2AJ1P, OR2AK2, OR2AP1, OR2AT4, OR2B2, OR2B3, OR2B6, OR2B11, OR2C1, OR2C3, OR2D2, OR2D3, OR2F1, OR2G2, OR2G3, OR2G6, OR2H1, OR2H2, OR2J2, OR2J3, OR2K2, OR2L2, OR2L8, OR2L13, OR2M2, OR2M4, OR2M7, OR2S2, OR2T1, OR2T2, OR2T5, OR2T6, OR2T8, OR2T10, OR2T11, OR2T27, OR2T34, OR2V2, OR2W1, OR2W3, OR2Y1, OR2Z1, OR3A1, OR3A2, OR3A3, OR3A4, OR4A5, OR4A15, OR4A16, OR4A47, OR4B1, OR4C3, OR4C5, OR4C6, OR4C11, OR4C12, OR4C13, OR4C15, OR4C16, OR4C46, OR4D1, OR4D2, OR4D5, OR4D6, OR4D9, OR4D10, OR4D11, OR4E2, OR4F3, OR4F5, OR4F6, OR4F14P, OR4F15, OR4G11P, OR4H12P, OR4K1, OR4K2, OR4K5, OR4K13, OR4K14, OR4K15, OR4K17, OR4L1, OR4M1, OR4N2, OR4N4, OR4N5, OR4P4, OR4Q3, OR4S1, OR4S2, OR4X1, OR4X2, OR5A1, OR5A2, OR5AC2, OR5AK2, OR5AK3P, OR5AN1, OR5AP2, OR5AR1, OR5AS1, OR5AU1, OR5B2, OR5B3, OR5B12, OR5B17, OR5B21, OR5C1, OR5D13, OR5D14, OR5D16, OR5D18, OR5F1, OR5H1, OR5H2, OR5H6, OR5H14, OR5I1, OR5J2, OR5K1, OR5K3, OR5K4, OR5L2, OR5M3, OR5M8, OR5M9, OR5M10, OR5M11, OR5P3, OR5R1, OR5T1, OR5T2, OR5T3, OR5V1, OR5W2, OR6A2, OR6B1, OR6B2, OR6C1, OR6C2, OR6C3, OR6C4, OR6C6, OR6C65, OR6C66P, OR6C68, OR6C70, OR6C74, OR6C75, OR6C76, OR6F1, OR6J1, OR6K2, OR6K3, OR6K6, OR6M1, OR6N1, OR6N2, OR6P1, OR6Q1, OR6S1, OR6T1, OR6V1, OR6X1, OR6Y1, OR7A3P, OR7A5, OR7A10, OR7A17, OR7C1, OR7C2, OR7D2, OR7D4, OR7E24, OR7G1, OR7G2, OR7G3, OR8A1, OR8B3, OR8B4, OR8B8, OR8B12, OR8D1, OR8D2, OR8D4, OR8G2, OR8G5, OR8H3, OR8I2, OR8J1, OR8J3, OR8K1, OR8K3, OR8K5, OR8S1, OR8U1, OR9A4, OR9G1, OR9G4, OR9I1, OR9K2, OR9Q1, OR9Q2, OR10A3, OR10A4, OR10A5, OR10A6, OR10A7, OR10AD1, OR10AG1, OR10C1, OR10D3, OR10D4P, OR10G2, OR10G3, OR10G4, OR10G6, OR10G7, OR10G9, OR10H2, OR10H4, OR10J1, OR10J3, OR10J5, OR10K1, OR10K2, OR10P1, OR10Q1, OR10R2, OR10S1, OR10T2, OR10V1, OR10W1, OR10X1, OR10Z1, OR11A1, OR11G2, OR11H4, OR11H6, OR11H12, OR11L1, OR12D2, OR12D3, OR13A1, OR13C2, OR13C3, OR13C4, OR13C8, OR13D1, OR13F1, OR13G1, OR13H1, OR13J1, OR14A2, OR14A16, OR14C36, OR14I1, OR14J1, OR14K1, OR14L1P, OR51A1P, OR51A4, OR51A7, OR51B2, OR51B4, OR51B5, OR51B6, OR51D1, OR51E1, OR51E2, OR51F1, OR51F2, OR51F5P, OR51G1, OR51G2, OR51H1, OR51I1, OR51I2, OR51L1, OR51M1, OR51Q1, OR51S1, OR51T1, OR51V1, OR52A1, OR52A4, OR52A5, OR52B2, OR52B4, OR52B6, OR52D1, OR52E2, OR52E4, OR52E5, OR52E8, OR52H1, OR52I2, OR52J3, OR52K2, OR52L2P, OR52M1, OR52N1, OR52N2, OR52N4, OR52N5, OR52P2P, OR52R1, OR52W1, OR52Z1P, OR56A1, OR56A3, OR56A4, OR56A5, OR56B1, OR56B2P, and OR56B4.

14. The method according to claim 1, wherein the receptors are human receptors.

15. The method according to claim 1, further comprising the step of evaluating a sensory quality of the predicted formulation.

16. The method according to claim 15, wherein the evaluation is carried out by sensory evaluation.

17. The method according to claim 1, comprising:
a step of identifying, for each of a plurality of targets, the two or more target substances and the combination ratio of receptor activity profiles of the target substances such that the target substances and the combination ratio provide the composite profile that approximates the target profile; and
a minimum set determination step of determining a predetermined number of synthetic elements and combination ratios of the synthetic elements such that the synthetic elements and the combination ratios approximate the identified composite profiles of the plurality of targets, and determining one or more target substances that constitute each of the synthetic elements.

18. The method according to claim 17, wherein, in the minimum set determination step, the composite profiles of the plurality of targets are decomposed, by nonnegative matrix factorization (NMF), into the predetermined number of synthetic elements and the combination ratios of the synthetic elements.

19. The method according to claim 1, wherein the target substances are blended at a blending ratio that is substantially identical to the combination ratio.

20. The method according to claim 19, wherein for each target substance, the blending ratio is 0.9 to 1.1 times the combination ratio.
